(19) United States
(12) Patent Application Publication
(10) Pub. No.: US 2006/0194215 A1
(43) Pub. Date: Aug. 31, 2006

(54) Methods, reagents and kits for reusing arrays

(76) Inventors: Mel N. Kronick, Palo Alto, CA (US); Eric M. Leproust, San Jose, CA (US); Douglas A. Amorese, Los Altos, CO (US)

Correspondence Address: AGILENT TECHNOLOGIES, INC.;Legal Department, DL 429, Intellectual Property Administration, P.O. Box 7599, Loveland, CO, 80537-0599, US

(21) Appl. No.: 11/069,731

(22) Filed: Feb. 28, 2005

(51) Int. Cl. C12Q 1/68

(52) U.S. Cl. 435/6

(57) ABSTRACT

Methods for stripping and reusing arrays are disclosed. Also disclosed are reagents and kits comprising control target molecules for determining the efficacy of a stripping condition to which an array has been exposed.

### BACKGROUND

Array assays between surface bound binding agents or probes and target molecules in solution may be used to detect the presence of particular biopolymers. The surface-bound probes may be oligonucleotides, peptides, polypeptides, proteins, antibodies, affibodies, aptamers or other molecules capable of binding with target molecules in solution. Such binding interactions are the basis for many of the methods and devices used in a variety of different fields, e.g., genomics (in sequencing by hybridization, SNP detection, differential gene expression analysis, identification of novel genes, gene mapping, finger printing, comparative genome hybridization, location analysis, etc.) and proteomics.

One typical array assay method involves biopolymeric probes immobilized in an array on a substrate such as a glass substrate or the like. A solution containing analytes that bind with the attached probes is placed in contact with the substrate, covered with another substrate to form an assay area and placed in an environmentally controlled chamber such as an incubator or the like. Usually, the targets in the solution bind to the complementary probes on the substrate to form a binding complex. The pattern of binding by target molecules to biopolymer probe features or spots on the substrate produces a pattern on the surface of the substrate and provides desired information about the sample. In most instances, the target molecules are labeled with a detectable tag such as a fluorescent tag, chemiluminescent tag or radioactive tag. The resultant binding interaction or complexes of binding pairs are then detected and read or interrogated, for example by optical means, although other methods may also be used. For example, laser light may be used to excite fluorescent tags, generating a signal only in those spots on the biochip that have a target molecule and thus a fluorescent tag bound to a probe molecule. This pattern may then be digitally scanned for computer analysis.

Biopolymer arrays can be fabricated by depositing previously obtained biopolymers (such as from synthesis or natural sources) onto a substrate, or by in situ synthesis methods. Methods of depositing obtained biopolymers include loading then touching a pin or capillary to a surface, such as described in U.S. Pat. No. 5,807,522 or deposition by firing from a pulse jet such as an inkjet head, such as described in PCT publications WO 95/25116 and WO 98/41531, and elsewhere. Such a deposition method can be regarded as forming each feature by one cycle of attachment (that is, there is only one cycle at each feature during which the previously obtained biopolymer is attached to the substrate). For in situ fabrication methods, multiple different reagent droplets are deposited by pulse jet or other means at a given target location in order to form the final feature (hence a probe of the feature is synthesized on the array substrate). Some in situ fabrication methods include those described in U.S. Pat. No. 5,449,754 for synthesizing peptide arrays, and in U.S. Pat. No. 6,180,351 and WO 98/41531 and the references cited therein for polynucleotides, and may also use pulse jets for depositing reagents.

An in situ method for fabricating a polynucleotide array typically follows: at each of the multiple different addresses at which features are to be formed, the same conventional iterative sequence used in forming polynucleotides from nucleoside reagents on a support by means of known chemistry. This iterative sequence can be considered as multiple ones of the following attachment cycle at each feature to be formed: (a) coupling an activated selected nucleoside (a monomeric unit) through a phosphite linkage to a functionalized support in the first iteration, or a nucleoside bound to the substrate (i.e. the nucleoside-modified substrate) in subsequent iterations; (b) optionally, blocking unreacted hydroxyl groups on the substrate bound nucleoside (sometimes referenced as “capping”); (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. The coupling can be performed by depositing drops of an activator and phosphoramidite at the specific desired feature locations for the array. A final deprotection step is provided in which nitrogenous bases and phosphate group are simultaneously deprotected by treatment with ammonium hydroxide and/or methylamine under known conditions. Capping, oxidation and deprotection can be accomplished by treating the entire substrate (“flooding”) with a layer of the appropriate reagent. The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in another flooding procedure in a known manner. Conventionally, a single pulse jet or other dispenser is assigned to deposit a single monomeric unit.

The foregoing chemistry of the synthesis of polynucleotides is described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. No. 4,458,066, U.S. Pat. No. 4,500,707, U.S. Pat. No. 5,153,319, U.S. Pat. No. 5,869,643, EP 0294196, and elsewhere. The phosphoramidite and phosphite triester approaches are most broadly used, but other approaches include the phosphodiester approach, the phosphotriester approach and the H-phosphonate approach. The substrates are typically functionalized to bond to the first deposited monomer. Suitable techniques for functionalizing substrates with such linking moieties are described, for example, in Southern, E. M., Maskos, U. and Elder, J. K., Genomics, 13, 1007-1017, 1992. In the case of array fabrication, different monomers and activator may be deposited at different addresses on the substrate during any one cycle so that the different features of the completed array will have different desired biopolymer sequences. One or more intermediate further steps may be required in each cycle, such as the conventional oxidation, capping and washing steps in the case of in situ fabrication of polynucleotide arrays (again, these steps may be performed in flooding procedure).

Further details of fabricating biopolymer arrays by depositing either previously obtained biopolymers or by the in situ method are disclosed in U.S. Pat. No. 6,242,266, U.S. Pat. No. 6,232,072, U.S. Pat. No. 6,180,351, and U.S. Pat. No. 6,171,797. In fabricating arrays by depositing previously obtained biopolymers or by the in situ method, typically each region on the substrate surface on which an array will be or has been formed (“array regions”) is completely exposed to one or more reagents. For example, in either method the array regions will often be exposed to one or more reagents to form a suitable layer on the surface that binds to both the substrate and biopolymer or biomonomer. In in situ fabrication the array regions will also typically be exposed to the oxidizing, deblocking, and optional capping reagents. Similarly, particularly in fabrication by depositing previously obtained biopolymers, it may be desirable to expose the array regions to a suitable blocking reagent to block locations on the surface at which there are no features from non-specifically binding to target.

It would be desirable to provide a means by which many arrays can be stripped and reused after detecting a binding pattern of target molecules on the array. It would also be desirable to be able to validate the stripping process prior to contacting with additional targets.

### SUMMARY OF THE INVENTION

In one embodiment, the invention relates to a method for facilitating reuse of arrays. In one aspect, the method comprises detecting binding of a first population of target molecules to an array; exposing the array to conditions for removing bound target molecules from the array; detecting binding of a second population of target molecules to the array, wherein said detecting includes detecting a first pattern of binding on the array which indicates to what extent removal of the bound targets has occurred. In another aspect, the first pattern comprises a pattern of binding of first stripping control target molecules present in the first population of target molecules on the array. In certain aspects, the first stripping control features are arranged to form a symbol, e.g., such as a number or a letter or other pattern, which can be then correlated with the particular binding procedure in which the stripping control target was applied.

In one embodiment, the step of detecting a pattern comprises determining the amount of binding of the first stripping control target molecules. In one aspect, the amount of binding is compared to a threshold amount. In certain aspects, when a difference is observed between the threshold amount and the amount of binding of the first stripping control target, binding data of the second population of molecules is associated with a data flag (e.g., by a reader of the array, whether a person or a scanner). For example, the data flag may be used to indicate that the step of removing bound targets has not proceeded satisfactorily and that the data in a subsequent binding assay should be discarded, or normalized to address for the residual binding from the first binding assay, that the array should be re-exposed to the conditions for removing bound target, and/or that settings of an array scanner for detecting signals corresponding to complexes formed in a subsequent binding assay should adjusted to detect signals higher than signals corresponding to complexes between the first stripping control target molecules and the first stripping control features after exposure to the stripping conditions.

For example, in another embodiment, a second population of target molecules is contacted to the array. The second population comprises second stripping control target molecules, which specifically bind to second stripping control features on the array. The second stripping control features are arranged in a second pattern, e.g., one that is different from the first pattern produced by the disposition of the first stripping control features. In one aspect, the second pattern is detected. If the first pattern as well as the second pattern is detected, i.e., signaling incomplete removal of second stripping control target molecules forming the first pattern, the array or data obtained from the array is associated with a data flag, signaling that data from the second hybridization should be discarded, treated as suspect, normalized to adjust signal resulting from residual binding of first target molecules, and/or that settings of an array scanner for detecting signals corresponding to the formation of complexes between the second target population and the array should be adjusted to detect signals higher than those signals from complexes generating the first pattern on the array which remain after exposing to the stripping conditions.

The process can be reiterated, e.g., the array may be exposed again to conditions for removing bound target molecules from the second population of target molecules from the array and the second pattern can be detected as a way to determine the efficacy of the stripping conditions. The array can be contacted with a third population of target nucleic acids comprising third stripping control target molecules which bind to third stripping control features disposed in a third pattern on the array which is different from the second pattern and in one aspect, also different from the first pattern. In one aspect, the third pattern is also a symbol, e.g., such as a letter or number or shape. The symbol can be recognizable after visual inspection of the array or an image of the array when labeled stripping control target molecules are bound to the pattern, or through the aid of a computer-based pattern-recognition algorithm.

In one embodiment, the symbol formed by the pattern can be correlated with the order in which target populations of molecules have been contacted with the array. For example, detecting a pattern resembling the number “1” in addition to the number “2” would indicate that there were residual complexes which had been incompletely removed from the first binding assay which are being detected after the second binding reaction.

In certain aspects, a pattern corresponding to a binding reaction is detected after exposing the array to conditions for removing target molecules from the array but prior to generating a subsequent pattern. For example, if signal from a first pattern is detected and exceeds a certain threshold level, the array can be re-exposed one or more times to the stripping conditions prior to exposing the array to the second population of target molecules and/or settings for an array scanner for detecting signals from the array can be adjusted to compensate for the residual signals from the first pattern on the array.

In another embodiment, performance control target molecules are additionally added to each sample being analyzed are employed to assess any degradation in the overall performance of the microarray (including, but not limited to signal to noise, dynamic range, linearity of response, and background) attributable to the re-use process. In one aspect, the performance control target molecules comprise a plurality of defined sequences present in known relative concentrations. Generally, these comprise sequences, e.g., such as adenovirus sequences, which are different from those of other target molecules (e.g., such as mammalian sequences) and bind to performance control features on the array, which may be, but are not necessarily disposed in a pattern on the array. If the overall performance of the array is not degraded during the stripping process, the relative ratios of performance control target molecules binding to the array should reflect the relative ratio of performance control target molecules present in the sample.

In one embodiment, the target molecules comprise biopolymers, such as nucleic acid molecules, polypeptides, carbohydrates, and the like. In another aspect, the target molecules comprise genomic DNA. In still a further aspect, the target molecules comprise RNA, cRNA, cDNA, and the like.

In another embodiment, the invention relates to kits comprising an array comprising a plurality of features, including first stripping control features disposed in a first pattern on the array. In one aspect, the kit includes a first set of stripping control targets for binding to first stripping control features disposed in a first pattern on the array. In another aspect, the kit further comprises a second set of stripping control targets for binding to second stripping control features disposed in a second pattern on the array. In one aspect, the first and second pattern are different. In another aspect, the first pattern comprises a symbol, such as a number or character or shape that can be recognized upon visual inspection of the array or an image of the array (e.g., after binding of labeled stripping control target molecules) or through the aid of a computer-based pattern recognition algorithm. In one aspect, the first and second patterns comprise different numbers.

In still another embodiment, the invention relates to an array comprising an identifier, wherein the identifier is associated with data relating to stripping conditions to which the array has been or should be exposed to. In one aspect, the identifier is associated with data relating to the disposition of stripping control probes for validating the efficacy of a stripping procedure on the array. In another aspect, the identifier comprises a data element comprising a remotely programmable memory. In still another aspect, the identifier comprises a bar code tag. The identifier can also be associated with data relating to array layout, array content, distribution of performance control features on the array, gene names associated with probes on the array, or data relating to genes or sequences associated with probes on the array (e.g., such as data relating to gene function, interactions with other molecules, encoded products and the like).

In a further embodiment, the invention relates to a device comprising one or more chambers comprising a means for exposing an array substrate to stripping conditions for removing bound target molecules to probe molecules on the array, wherein the device further comprises or is associated with an identifier reader for reading an identifier on an array. In one aspect, the means for exposing comprises an inlet in the chamber which communicates with a reservoir comprising a fluid for stripping the array. In another aspect, the means comprises a heating element for raising the temperature of a fluid within the chamber to a temperature effective for stripping the array. In a further aspect, the chamber further comprises an outlet for removing a fluid from the chamber. In yet another aspect, the chamber comprises an additional inlet for introducing fluids for washing unbound target molecules from an array or for introducing target molecules for contacting with the array. In still other aspects, the chamber further comprises an inlet for introducing a liquid or gaseous fluid for drying the array.

In certain embodiments, the device further comprises an additional chamber comprising an inlet for introducing fluids for washing unbound target molecules from the array or for introducing target molecules for contacting with the array. In certain embodiments, the device further comprises a means for moving an array substrate from one chamber to another, such as a robotic transfer station or other like mechanism. In one aspect, a chamber of the device is removable and configured for placement in a scanner. In still other aspects, the chamber is configured to receive an array holder or array assembly for containing the array substrate and the array holder or assembly is configured to be placed in a scanner for reading the array.

In one aspect, the device comprises a plurality of inlets which communicate independently with one or more chambers of the device.

In certain aspects, the device also comprises a processor for controlling movements of fluids and/or conditions within the one or more chambers. In certain aspects, the processor communicates with a memory for storing data relating to stripping conditions and/or array performance after stripping. In one aspect, the device further comprises a user interface for communicating with the processor and for displaying one or more stripping procedures. In another aspect, the stripping procedure is associated with an assay type and the stripping procedure is executed by the device in response to a user selecting the assay type displayed on the user interface.

In still another aspect, the processor further communicates with a scanner, receiving input from the scanner relating to signal intensity at stripping control features after a stripping procedure and can implement a protocol for re-exposing an array to stripping conditions based on the signal intensity at the control features.

In a further aspect, in response to reading an identifier on an array within a chamber of the device, the device executes a procedure (e.g., such as a stripping procedure) associated with the identifier in a memory accessed by the processor.

In still a further aspect, in response to reading an identifier on the array, the device provides an output relating to procedures to which the array has been exposed within the device. The output can be displayed on a user interface in communication with the device, which may be remote from the device. In additional aspects, the processor accesses a memory storing data relating to array identifiers and data associated with the array. The data can include, but is not limited to, the nature of probes on the array, a pattern in which one or more stripping control probe features is disposed, the disposition performance control features on the array, the ratio of performance control target molecules in a sample, the nature of a procedure or condition to which the array has been or should be exposed, binding conditions to which the array has been exposed, washing conditions to which the array has been exposed, and/or stripping conditions to which the array has been exposed. In one aspect, updated data is provided to the processor prior to and/or after exposing the array to a fluid in one or more chambers. In another aspect, when a user selects a procedure to be executed by the device or alters parameters of a procedure to which an array is exposed, information relating to that altered procedure and/or parameters is automatically stored in a memory accessible by the processor and is associated with an identifier on the array.

In another embodiment, the invention also relates to a computer program product comprising instructions for executing operations of a chamber in which an array is being stripped, based on inputted data relating to a pattern of stripping control target molecules bound to stripping control probe features on an array which has been exposed to a stripping procedure. In one aspect, the computer program product comprises instructions to compute performance of the array (e.g., by assigning a performance value, such as “acceptable” or “not acceptable,” based on a predetermined threshold) based on signal from performance control target molecules hybridized to performance control probes on an array which has been exposed to a stripping procedure, for example, in order to determine the effects on performance which are attributable to the stripping of a microarray. The computer program product may assess inputs, related to one or more of stripping conditions, hybridization conditions, and/or washing conditions, and can associate the conditions with a value for a signal associated with stripping control probe: stripping control target complexes obtained after stripping, e.g., as a way of identifying quality control metrics for stripping.

In a further embodiment, the invention relates to a computer memory comprising data associating an identifier on an array with a stripping protocol to which the array has been or should be exposed. In one aspect, a processor communicates with the memory to execute instructions relating to the stripping protocol.

The invention also relates to a method of certifying an array for reuse, comprising providing a user of an array (e.g., such as a customer of an array provider) with a warranty or certification that data obtained from the array after a stripping procedure will be comparable or insubstantially different from the data that would be obtained from the array if the stripping procedure had not been performed. In one aspect, the method of certifying comprises determining that a user has followed an accepted stripping protocol validated by the supplier of the array. In another, aspect, the accepted stripping protocol is programmed into a device used by a user for stripping the array. In yet another aspect, the device can provide a message (such as an email message) to a supplier confirming that a validated stripping process has been followed.

### BRIEF DESCRIPTION OF THE DRAWING

The objects and features of the invention can be better understood with reference to the following detailed description and accompanying drawings. The Figures shown herein are not necessarily drawn to scale, with some components and features being exaggerated for clarity.

FIGS. 1A-C are schematic diagrams showing multiple views of a single array. The circles represent features on the array. Filled in circles represent signals from complexes formed between stripping control probes on the array and complementary stripping control target molecules. FIG. 1A shows the array binding to a first population of target molecules comprising first stripping control target molecules binding to first stripping control probe features disposed in a pattern resembling the number “1” on the array. FIG. 1B shows the same array after exposure to stripping conditions and to a second population of target molecules comprising second stripping control target molecules which bind to second stripping control probe features disposed on a second pattern resembling the number “2” on the array. FIG. 1C shows an array in which the first population of first stripping control target molecules remains detectable after exposing the array to stripping conditions and hybridizing the array to the second population of stripping control target molecules.

### DESCRIPTION OF THE INVENTION

Before describing the present invention in detail, it is to be understood that this invention is not limited to specific compositions, method steps, or equipment, as such may vary. It is also to be understood that the terminology used herein is for the purpose of describing particular embodiments only, and is not intended to be limiting. Methods recited herein may be carried out in any order of the recited events that is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

Unless defined otherwise below, all technical and scientific terms used herein have the same meaning as commonly understood by one of ordinary skill in the art to which this invention belongs. Still, certain elements are defined herein for the sake of clarity.

All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It must be noted that, as used in this specification and the appended claims, the singular forms “a”, “an” and “the” include plural referents unless the context clearly dictates otherwise. Thus, for example, reference to “a biopolymer” includes more than one biopolymer, and reference to “a voltage source” includes a plurality of voltage sources and the like.

Definitions

The following definitions are provided for specific terms that are used in the following written description.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “mRNA” means messenger RNA.

A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Nucleotide sub-units of deoxyribonucleic acids are deoxyribonucleotides, and nucleotide sub-units of ribonucleic acids are ribonucleotides.

An “oligonucleotide” generally refers to a nucleotide multimer of about 10 to 100 nucleotides in length, while a “polynucleotide” includes a nucleotide multimer having any number of nucleotides.

A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand features, or even more than one hundred thousand features, in an area of less than 20 cm² or even less than 10 cm². For example, features may have widths (that is, diameter, for a round spot) in the range of from about 10 μm to about 1.0 cm. In other embodiments each feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges. A given feature is made up of chemical moieties, e.g., nucleic acids, that bind to (e.g., hybridize to) the same target (e.g., target nucleic acid), such that a given feature corresponds to a particular target. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). The target for which each feature is specific is, in representative embodiments, known. An array feature is generally homogenous in composition and concentration and the features may be separated by intervening spaces (although arrays without such separation can be fabricated).

In the case of an array, the “target” will be referenced as a moiety in a mobile phase (typically fluid), to be detected by probes (“target probes”) which are bound to the substrate at the various regions. However, either of the “target” or “target probes” may be the one which is to be detected by the other (thus, either one could be an unknown mixture of polynucleotides to be detected by binding with the other). “Addressable sets of probes” and analogous terms refer to the multiple regions of different moieties supported by or intended to be supported by the array surface.

The term “sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiments, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence. In certain aspects, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

By “genomic source” is meant the initial nucleic acids that are used as the original nucleic acid source from which the probe nucleic acids are produced, e.g., as a template in the nucleic acid amplification and/or labeling protocols.

If a surface-bound polynucleotide or probe “corresponds to” a chromosomal region, the polynucleotide usually contains a sequence of nucleic acids that is unique to that chromosomal region. Accordingly, a surface-bound polynucleotide that corresponds to a particular chromosomal region usually specifically hybridizes to a labeled nucleic acid made from that chromosomal region, relative to labeled nucleic acids made from other chromosomal regions.

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

The phrase “oligonucleotide bound to a surface of a solid support” or “probe bound to a solid support” or a “target bound to a solid support” refers to an oligonucleotide or mimetic thereof, e.g., PNA, LNA or UNA molecule that is immobilized on a surface of a solid substrate, where the substrate can have a variety of configurations, e.g., a sheet, bead, particle, slide, wafer, web, fiber, tube, capillary, microfluidic channel or reservoir, or other structure. In certain embodiments, the collections of oligonucleotide elements employed herein are present on a surface of the same planar support, e.g., in the form of an array. It should be understood that the terms “probe” and “target” are relative terms and that a molecule considered as a probe in certain assays may function as a target in other assays.

As used herein, a “test nucleic acid sample” or “test nucleic acids” refer to nucleic acids comprising sequences whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed.

As used herein, a “reference nucleic acid sample” or “reference nucleic acids” refers to nucleic acids comprising sequences whose quantity or degree of representation (e.g., copy number) or sequence identity is known. Similarly, “reference genomic acids” or a “reference genomic sample” refers to genomic nucleic acids comprising sequences whose quantity or degree of representation (e.g., copy number) or sequence identity is known. A “reference nucleic acid sample” may be derived independently from a “test nucleic acid sample,” i.e., the samples can be obtained from different organisms or different cell populations of the sample organism. However, in certain embodiments, a reference nucleic acid is present in a “test nucleic acid sample” which comprises one or more sequences whose quantity or identity or degree of representation in the sample is unknown while containing one or more sequences (the reference sequences) whose quantity or identity or degree of representation in the sample is known. The reference nucleic acid may be naturally present in a sample (e.g., present in the cell from which the sample was obtained) or may be added to or spiked in the sample.

If a surface-bound polynucleotide or probe “corresponds to” a chromosome, the polynucleotide usually contains a sequence of nucleic acids that is unique to that chromosome. Accordingly, a surface-bound polynucleotide that corresponds to a particular chromosome usually specifically hybridizes to a labeled nucleic acid made from that chromosome, relative to labeled nucleic acids made from other chromosomes. Array features, because they usually contain surface-bound polynucleotides, can also correspond to a chromosome.

A “non-cellular chromosome composition” is a composition of chromosomes synthesized by mixing pre-determined amounts of individual chromosomes. These synthetic compositions can include selected concentrations and ratios of chromosomes that do not naturally occur in a cell, including any cell grown in tissue culture. Non-cellular chromosome compositions may contain more than an entire complement of chromosomes from a cell, and, as such, may include extra copies of one or more chromosomes from that cell. Non-cellular chromosome compositions may also contain less than the entire complement of chromosomes from a cell.

“Hybridizing” and “binding”, with respect to polynucleotides, are used herein interchangeably.

The term “duplex Tₘ” refers to the melting temperature of two oligonucleotides that have formed a duplex structure.

The term “predetermined” refers to an element whose identity or composition is known prior to its use. For example, a “predetermined temperature” is a temperature that is specified as a given temperature prior to use. An element may be known by name, sequence, molecular weight, its function, or any other attribute or identifier. As used herein, “automatic”, automatically”, or other like term references a process or series of steps that occurs without further intervention by the user, typically as a result of a triggering event provided or performed by the user.

As used herein, the term “signal” refers to the detectable characteristic of a detectable molecule. Exemplary detectable characteristics include, but are not limited to: a change in the light adsorption characteristics of a reaction solution resulting from enzymatic action of an enzyme attached to a labeling probe acting on a substrate; the color or change in color of a dye; fluorescence; phosphorescence; radioactivity; or any other indicia that can be detected and/or quantified by a detection system being used.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

A “plastic” is any synthetic organic polymer of high molecular weight (for example at least 1,000 grams/mole, or even at least 10,000 or 100,000 grams/mole.

“Flexible” with reference to a substrate or substrate web (including a housing or one or more housing component such as a housing base and/or cover), references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C. “Rigid” refers to a substrate (including a housing or one or more housing component such as a housing base and/or cover) which is not flexible, and is constructed such that a segment about 2.5 by 7.5 cm retains its shape and cannot be bent along any direction more than 60 degrees (and often not more than 40, 20, 10, or 5 degrees) without breaking.

When one item is indicated as being “remote” from another, this descriptor indicates that the two items are at least in different buildings, and may be at least one mile, ten miles, or at least one hundred miles apart. When different items are indicated as being “local” to each other they are not remote from one another (for example, they can be in the same building or the same room of a building). “Communicating”, “transmitting” and the like, of information reference conveying data representing information as electrical or optical signals over a suitable communication channel (for example, a private or public network, wired, optical fiber, wireless radio or satellite, or otherwise). Any communication or transmission can be between devices that are local or remote from one another. “Forwarding” an item refers to any means of getting that item from one location to the next, whether by physically transporting that item or using other known methods (where that is possible) and includes, at least in the case of data, physically transporting a medium carrying the data or communicating the data over a communication channel (including electrical, optical, or wireless). “Receiving” something means it is obtained by any possible means, such as delivery of a physical item (for example, an array or array carrying package). When information is received it may be obtained as data as a result of a transmission (such as by electrical or optical signals over any communication channel of a type mentioned herein), or it may be obtained as electrical or optical signals from reading some other medium (such as a magnetic, optical, or solid state storage device) carrying the information. However, when information is received from a communication it is received as a result of a transmission of that information from elsewhere (local or remote).

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. Items of data are “linked” to one another in a memory when a same data input (for example, filename or directory name or search term) retrieves those items (in a same file or not) or an input of one or more of the linked items retrieves one or more of the others. In particular, when an array layout is “linked” with an identifier for that array, then an input of the identifier into a processor which accesses a memory carrying the linked array layout retrieves the array layout for that array.

A “computer”, “processor” or “processing unit” are used interchangeably and each references any hardware or hardware/software combination which can control components as required to execute recited steps. For example a computer, processor, or processor unit includes a general purpose digital microprocessor suitably programmed to perform all of the steps required of it, or any hardware or hardware/software combination, which will perform those, or equivalent steps. Programming may be accomplished, for example, from a computer readable medium carrying necessary program code (such as a portable storage medium) or by communication from a remote location (such as through a communication channel).

A “memory” or “memory unit” refers to any device that can store information for retrieval as signals by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

An array “assembly” includes a substrate and at least one chemical array on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”.

“Reading” signal data from an array refers to the detection of the signal data (such as by a detector) from the array. This data may be saved in a memory (whether for relatively short or longer terms).

A “package” is one or more items (such as an array assembly optionally with other items) all held together (such as by a common wrapping or protective cover or binding). Normally the common wrapping will also be a protective cover (such as a common wrapping or box), which will provide additional protection to items contained in the package from exposure to the external environment. In the case of just a single array assembly a package may be that array assembly with some protective covering over the array assembly (which protective cover may or may not be an additional part of the array unit itself).

It will also be appreciated that throughout the present application, that words such as “cover”, “base” “front”, “back”, “top”, “upper”, and “lower” are used in a relative sense only.

“May” refers to optionally.

When two or more items (for example, elements or processes) are referenced by an alternative “or”, this indicates that either could be present separately or any combination of them could be present together except where the presence of one necessarily excludes the other or others.

In one embodiment, the invention relates to methods for reusing a chemical array. Preferred substrate materials for forming arrays are those that provide physical support for the chemical compounds that are deposited on the substrate surface or synthesized on the surface in situ from subunits. The materials should be of such a composition that they endure the conditions of a deposition process and/or an in situ synthesis and/or any subsequent treatment or handling or processing that may be encountered in the use of the particular array, including stripping as described further below.

Typically, the substrate material is transparent, i.e., the substrate material permits signal from features on the surface of the substrate to pass through it without substantial attenuation and also permits any interrogating radiation to pass through it without substantial attenuation, e.g., without a loss of more than 40% or without a loss of more than 30%, 20% or 10%, of signal. The interrogating radiation and signal may, for example, be visible, ultraviolet or infrared light. However, it should be noted that the nature of the transparency of the substrate is somewhat dependent on the nature of the scanner employed to read the substrate surface. Some scanners work with opaque or reflective substrates.

The material may be naturally occurring or synthetic or modified naturally occurring. Substrates can be rigid, flexible, or semi-rigid. Suitable substrate materials include, but are not limited to: silicon, silica or glass, mirrored surfaces, laminates, ceramics, opaque plastics, and the like. Natural or synthetic polymeric materials can also be used, e.g., such as cellulosic materials and materials derived from cellulose, such as fiber-containing papers, e.g., filter paper, chromatographic paper, etc., nitrocellulose, cellulose acetate, poly(vinyl chloride), polyamides, polyacrylamide, polyacrylate, polymethacrylate, polyesters, polyolefins, polyethylene, polytetrafluoroethylene, polypropylene, poly(4-methylbutene), polystyrene, poly(ethylene terephthalate), nylon, poly(vinyl butyrate), cross-linked dextran, agarose, etc.; either used by themselves or in conjunction with other materials. Additionally, the substrate can be hydrophilic or capable of being rendered hydrophilic or can comprise both hydrophilic and hydrophobic regions.

Suitable substrates may exist, for example, as sheets, tubing, spheres, containers, pads, slices, films, plates, slides, strips, disks, etc. The substrate can be flat, or can take on alternative surface configurations. In one aspect, the substrate is a flat glass substrate, such as a conventional microscope glass slide. In one embodiment, multiple arrays of chemical compounds are synthesized on a sheet, which is then singulated, such as, e.g., cut by breaking along score lines, into single array slides. The sheet of material may be of any convenient size depending on the nature of the equipment used, production lot size, productive efficiencies, production throughput demands, and so forth. In some embodiments, the sheet of material is usually about 5, about 6, about 7, about 8, about 9, about 10, about 11, about 12, about 13 inches in length and about 5, about 6, about 7, about 8, about 9, about 10, about 11, about 12, about 13 inches in width so that the sheet may be divided into multiple single array substrates having the dimensions indicated below. The thickness of the sheet may be less than 1 cm, or even less than 5 mm, 2 mm, 1 mm, or in some embodiments even less than 0.5 mm or 0.2 mm. The thickness of the substrate is about 0.01 mm to 5.0 mm, usually from about 0.1 mm to 2 mm and more usually from about 0.2 to 1. In a specific embodiment by way of illustration and not limitation, a wafer that is 6.25 inches by 6 inches by 1 mm is employed.

An individual or single substrate can be produced by dividing the sheet, for example, along predetermined lines. The individual support usually has a single array of chemical compounds that have been synthesized or deposited on a surface when the individual support was part of the sheet. The dimensions of the individual support are determined by the number of features in the array on the surface of the support, the intended use of the support, e.g., in conducting assays involving the chemical compounds on the surface of the support, ease of manual and automated handling steps, and so forth.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm² e.g., less than about 5 cm², including less than about 1 cm², less than about 1 mm², e.g., 100 μ², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features).

Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

The surface of the material onto which the chemical compounds are deposited or formed may be smooth or substantially planar, or have irregularities, such as depressions or elevations. The surface may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof (for example, peptide nucleic acids and the like); polysaccharides, phospholipids, polyurethanes, polyesters, polycarbonates, polyureas, polyamides, polyethylene amines, polyarylene sulfides, polysiloxanes, polyimides, polyacetates, and the like, where the polymers may be hetero- or homo-polymeric, and may or may not have separate functional moieties attached thereto (for example, conjugated). Various further modifications to the particular embodiments described above are, of course, possible. Accordingly, the present invention is not limited to the particular embodiments described in detail above.

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

In some embodiments, one area of the individual support that is a non-interfeature area or a portion of a border or a combination thereof comprises an identifier such as, e.g., a bar code. It is often desirable to have some type of identification on the array substrate that allows matching a particular array to layout information, since array layout information in some form is used to meaningfully interpret the information obtained from interrogating the array. Unique identifiers and their generation have been previously described, such as in U.S. Pat. No. 5,812,793, U.S. Pat. No. 5,404,523, and the references cited therein. Such unique identifiers (often referred to as “Globally Unique Identifiers” or “GUIDs”, or “Universally Unique Identifiers” or “UUIDs”) can, for example, include a network card identification, which is specific to that card, along with a time and local counter number, and other components.

Use of such unique identifiers in association with array layouts or distinct copies of the same layout generated at the same or different locations would virtually eliminate the possibility of the same identifier being associated with different array layouts or distinct copies of the same layout. However, such unique identifiers typically require 128 bit data string. A string of such length when written, for example, as a bar code, typically takes up about 3 to 4 cm, which is not consistent with the size of the individual supports disclosed herein. However, U.S. Pat. No. 6,180,351 describes an approach wherein a second identifier is employed of shorter length than a corresponding unique identifier, and which is associated in some manner with the unique identifier. The second identifier is placed on the individual support in a location not occupied by features of the array. The disclosure of U.S. Pat. No. 6,180,351 is incorporated herein by reference in its entirety.

The number of nucleic acid features of the initial or precursor array may vary, where the number of features present on the surface of the array may be at least 2, 5, or 10 or more such as at least 20 and including at least 50, where the number may be as high as about 100, as about 500, as about 1000, as about 5000, as about 10000 or higher, e.g., 25,000 or higher, 50,000 or higher, 100,000 or higher, 500,000 or higher, 1,000,000 or higher, etc. In some embodiments, the subject arrays have a density ranging from about 1000 to about 10,000 features/cm², such as from about 2,000 to about 10,000 features/cm 2, including from about 2,000 to about 5,000 features/cm². In certain of these embodiments, the density of the single-stranded nucleic acids may range from about 10⁻³ to about 1 pmol/mm², such as from about 10⁻² to about 0.1 pmol/mm², including from about 5×10⁻² to about 0.1 pmol/mm².

Each array may cover an area of less than about 100 cm², or even less than about 50 cm², 10 cm² or 1 cm². In many embodiments, the substrate carrying the one or more arrays will be shaped generally as a rectangular solid (although other shapes are possible and the substrate could be porous, have porous regions or have surfaces which are not-substantially planar), having a length of more than about 4 mm and less than about 1 m, usually more than about 4 mm and less than about 600 mm, more usually less than about 400 mm; a width of more than about 4 mm and less than about 1 m, usually less than about 500 mm and more usually less than about 400 mm; and a thickness of more than about 0.01 mm and less than about 5.0 mm, usually more than about 0.1 mm and less than about 2 mm and more usually more than about 0.2 and less than about 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least about 20%, or about 50% (or even at least about 70%, 90%, or 95%), of the illuminating light incident on the substrate as may be measured across the entire integrated spectrum of such illuminating light or alternatively at 532 nm or 633 nm.

In certain aspects, the array provides probes for screening or scanning a genome of an organism and comprises probes from a plurality of regions of the genome. In one aspect, the array comprises probe sequences for scanning an entire chromosome arm, wherein probes targets are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In another aspect, the array comprises probes sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes may also vary, and may be uniform, such that the spacing is substantially the same between sampled regions, or non-uniform, as desired. An assay performed at low resolution on one array, e.g., comprising probe targets separated by larger distances, may be repeated at higher resolution on another array, e.g., comprising probe targets separated by smaller distances.

Of interest, in constructing the arrays, are both coding and non-coding genomic regions, whereby “coding region” refers to a region comprising one or more exons that is transcribed into an mRNA product and from there translated into a protein product, while by non-coding region is meant any sequences outside of the exon regions, where such regions may include regulatory sequences, e.g., promoters, enhancers, untranslated but transcribed regions, introns, origins of replication, telomeres, etc. In certain embodiments, one can have at least some of the targets directed to non-coding regions and others directed to coding regions. In certain embodiments, one can have all of the targets directed to non-coding sequences. In certain embodiments, one can have all of the targets directed to coding sequences. In certain other aspects, individual probes comprise sequences that do not normally occur together, e.g., to detect gene rearrangements, for example.

In some embodiments, at least 5% of the polynucleotide probes on the solid support hybridize to regulatory regions of a nucleotide sample of interest while other embodiments may have at least 30% of the polynucleotide probes on the solid support hybridize to exonic regions of a nucleotide sample of interest. In yet other embodiments, at least 50% of the polynucleotide probes on the solid support hybridize to intergenic (e.g., non-coding) regions of a nucleotide sample of interest. In certain aspects, probes on the array represent random selection of genomic sequences (e.g., both coding and noncoding). However, in other aspects, particular regions of the genome are selected for representation on the array, e.g., such as CpG islands, genes belonging to particular pathways of interest or whose expression and/or copy number are associated with particular physiological responses of interest (e.g., disease, such a cancer, drug resistance, toxological responses and the like). In certain aspects, where particular genes are identified as being of interest, intergenic regions proximal to those genes are included on the array along with, optionally, all or portions of the coding sequence corresponding to the genes. In one aspect, at least about 100 bp, 500 bp, 1,000 bp, 5,000 bp, 10,000 kb or even 100,000 kb of genomic DNA upstream of a transcriptional start site is represented on the array in discrete or overlapping sequence probes. In certain aspects, at least one probe sequence comprises a motif sequence to which a protein of interest (e.g., such as a transcription factor) is known or suspected to bind.

In certain aspects, repetitive sequences are excluded as probes on the arrays. However, in another aspect, repetitive sequences are included.

The choice of nucleic acids to use as probes may be influenced by prior knowledge of the association of a particular chromosome or chromosomal region with certain disease conditions. International Application WO 93/18186 provides a list of exemplary chromosomal abnormalities and associated diseases, which are described in the scientific literature. Alternatively, whole genome screening to identify new regions subject to frequent changes in copy number can be performed using the methods of the present invention discussed further below.

In some embodiments, previously identified regions from a particular chromosomal region of interest are used as probes. In certain embodiments, the array can include probes which “tile” a particular region (e.g., which have been identified in a previous assay or from a genetic analysis of linkage), by which is meant that the probes correspond to a region of interest as well as genomic sequences found at defined intervals on either side, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled array tailored to the initially identified region to further assay the region at a higher resolution, e.g., in an iterative protocol.

In certain aspects, the array includes probes to sequences associated with diseases associated with chromosomal imbalances for prenatal testing. For example, in one aspect, the array comprises probes complementary to all or a portion of chromosome 21 (e.g., Down's syndrome), all or a portion of the X chromosome (e.g., to detect an X chromosome deficiency as in Turner's Syndrome) and/or all or a portion of the Y chromosome Klinefelter Syndrome (to detect duplication of an X chromosome and the presence of a Y chromosome), all or a portion of chromosome 7 (e.g., to detect William's Syndrome), all or a portion of chromosome 8 (e.g., to detect Langer-Giedon Syndrome), all or a portion of chromosome 15 (e.g., to detect Prader-Willi or Angelman's Syndrome, all or a portion of chromosome 22 (e.g., to detect Di George's syndrome).

Other “themed” arrays may be fabricated, for example, arrays including whose duplications or deletions are associated with specific types of cancer (e.g., breast cancer, prostate cancer and the like). The selection of such arrays may be based on patient information such as familial inheritance of particular genetic abnormalities. In certain aspects, an array for scanning an entire genome is first contacted with a sample and then a higher-resolution array is selected based on the results of such scanning.

Themed arrays also can be fabricated for use in gene expression assays, for example, to detect expression of genes involved in selected pathways of interest, or genes associated with particular diseases of interest.

In one embodiment, a plurality of probes on the array are selected to have a duplex Tₘ within a predetermined range. For example, in one aspect, at least about 50% of the probes have a duplex Tₘ within a temperature range of about 75° C. to about 85° C. In one embodiment, at least 80% of said polynucleotide probes have a duplex Tₘ within a temperature range of about 75° C. to about 85° C., within a range of about 77° C. to about 83° C., within a range of from about 78° C. to about 82° C. or within a range from about 79° C. to about 82° C. In one aspect, at least about 50% of probes on an array have range of Tₘ's of less than about 4° C., less then about 3° C., or even less than about 2° C., e.g., less than about 1.5° C., less than about 1.0° C. or about 0.5° C.

The probes on the microarray, in certain embodiments have a nucleotide length in the range of at least 30 nucleotides to 200 nucleotides, or in the range of at least about 30 to about 150 nucleotides. In other embodiments, at least about 50% of the polynucleotide probes on the solid support have the same nucleotide length, and that length may be about 60 nucleotides.

In certain aspects, longer polynucleotides may be used as probes. In addition to the oligonucleotide probes described above, cDNAs, or inserts from phage BACs (bacterial artificial chromosomes) or plasmid clones, can be arrayed. Probes may therefore also range from about 201-5000 bases in length, from about 5001-50,000 bases in length, or from about 50,001-200,000 bases in length, depending on the platform used. If other polynucleotide features are present on a subject array, they may be interspersed with, or in a separately-hybridizable part of the array from the subject oligonucleotides.

In still other aspects, probes on the array comprise at least coding sequences, e.g., for use in a gene expression assay. In one aspect, probes represent sequences from an organism such as Drosophila melanogaster, Caenorhabditis elegans, yeast, zebrafish, a mouse, a rat, a primate, a human, etc. In certain aspects, probes representing sequences from different organisms are provided on a single substrate, e.g., on a plurality of different arrays.

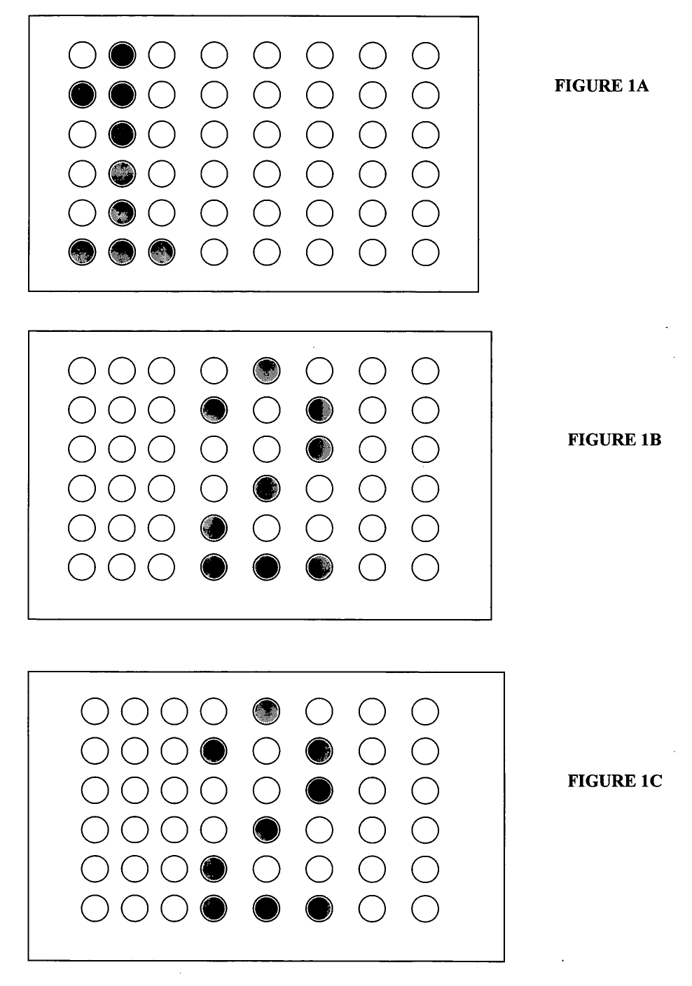

Any of a variety of geometries of features on a substrate may be used. In one aspect, features of an array are arranged in rectilinear rows and columns. In on aspect, an array comprises at least two stripping control features comprising stripping control probes disposed in a pattern. A pattern may comprise linear or curved elements or combinations thereof. In one aspect, stripping control features are disposed in a pattern, which is recognizable by humans or by a computer-based pattern recognition algorithm as a symbol or character when the stripping control features are complexed with stripping control probes. For example, the stripping control features may form the numeral “1” or “2” when complexed with stripping control probes as shown in FIGS. 1A-C. In another aspect, an array comprises at least two patterns of stripping control features, disposed in different locations on the array. In still another aspects, the different locations are non-overlapping, e.g., one pattern does not comprise any features that contribute to another pattern. In a further aspect, the patterns are different from each other, i.e., distinguishable by appearance, regardless of their position on the array. For example, each pattern may form a different symbol or character, such as different numbers.

In another embodiment, performance control target molecules also are added to each sample being analyzed to assess any degradation in the overall performance of the microarray (including, but not limited to signal to noise, dynamic range, linearity of response, and background) attributable to the re-use process. Such performance control target molecules comprise sequences that bind, under the employed hybridization conditions, to performance control probes with complementary sequences at pre-defined positions within the array layout. In one aspect, a plurality of performance control probe features are provided on the array which are complementary to a plurality of performance control target molecules. The plurality of performance control target molecules comprise defined sequences present in known ratios and the complexes formed between performance control probe molecules and performance control targets should be present in the same relative ratios, thereby providing a mechanism to assess the performance of the array after the stripping process. In one aspect, a performance control probe sequence is complementary to an adenovirus type 5 E1a sequence (e.g., nucleotides 560-972 or a subsequence thereof).

Performance control probe features, may be, but are not necessarily disposed in a pattern on the array. Although in one aspect, a pattern of performance control features is provided on the array, wherein a plurality of the features in the pattern can comprise different sequences. In another aspect, the performance control target molecules bind at different relative concentrations at the different performance control features to provide non-uniform signal intensities in the pattern that is formed, e.g., a portion of the pattern may produce a signal that has a different intensity from another portion of the pattern. In one aspect, a gradient of signal intensities form from one end of the pattern to another. However, in still another aspect, the performance control probe features are not disposed in any pattern on the array. While the stripping control probes in any given pattern comprise substantially identical or identical sequences, the performance control features can comprise a plurality of different sequences, even within the same pattern if they are disposed in a pattern.

In certain aspects, a plurality of arrays are provided on a substrate. In one aspect, the plurality of arrays comprises an array that is dedicated for use in validating the efficacy of a stripping process (e.g., comprising only stripping control probe features and optionally, performance control probe features). For example, the validation array can include stripping control features disposed in one or more patterns and, optionally performance control features which may or may not be disposed in a pattern. However, in other aspects, the stripping control features are part of an array where other features are provided to test for, e.g., gene expression, nucleic acid copy number, binding to regulatory proteins (e.g., including, but not limited to: transcription factors, chromatin, chromatin binding and/or modifying proteins, centromere binding proteins, telomere binding proteins, and/or proteins involved in RNA transcription, DNA replication, repair, recombination, modification, etc.).

In one embodiment, the stripping control probes associated with particular assays (e.g., a first and second assay) comprise substantially the same base composition (and in certain aspects, identical base composition; although not necessarily the same sequence). In one aspect, the order of bases in different stripping control probes (e.g., probes disposed in a first and second pattern on the array) are different. In another aspect, stripping control probes comprise high Tₘ's, e.g., of the Tₘ's of probes that are bound on the array, the stripping control probes comprise the highest 10%, the highest 5%, the highest 2%, the highest 1%, the highest 0.5% or the highest 0.1% of the Tₘ's. In still another aspect, stripping control targets bound to control probes on the array are among the strongest binding populations of nucleic acid molecules washed off in a stripping procedures. In a further aspect, stripping control target molecules bind to stripping control probes on the array even at low concentrations (e.g., nanomolar to picomolar concentrations). In still a further aspect, stripping control targets are present at a quantity that results in a signal of about 0.5-fold over background (e.g., a signal obtained from an interfeature or interarray area on an array substrate), less than about 1-fold over background, less than about 1.5-fold over background, less than about 2-fold over background, less than about 3-fold over background, or less than about 5-fold over background. In one aspect, the nature of the stripping control probe is independent of the type of assay. For example, a stripping control probe used to monitor stripping efficacy in a gene expression assay could also be used to validate stripping in a CGH assay or a location analysis assay. However, in another aspect, stripping control probes are selected to provide signals in the presence of an appropriate amount of control target that are less than about 5-fold background under the particular assay conditions used (e.g., for a gene expression assay, for a CGH assay or for a location analysis assay).

In certain aspects, replicates of stripping control features are provided. Different patterns may be selected for stripping control features associated with a given population of target stripping control molecules so long as patterns are distinguishable from one assay to another. For example, a first target population comprising first stripping control molecules may bind to features disposed in patterns “1,” “2” and “3” while a second target population comprising second stripping control molecules may bind to control features disposed in patterns “4,” “5” and “6.”

In one aspect, stripping control features forming a pattern on the array are separated from other features by atypical sizes of interfeature areas. In another aspect, stripping control features are bounded by negative control features, such as features that are not expected to bind to any targets, e.g., such features include probes forming intramolecular bonds or hairpins, reversed polarity sequences, non-natural bases, and/or probes which have been empirically observed or are predicted to bind minimally to target sequences. In still another aspect, stripping control features are bounded by target features corresponding to target molecules expected to be present at different copy numbers compared to control targets molecules. In a further aspect, as discussed above, stripping control features are physically segregated from other features, e.g., in distinct arrays or subarrays on a substrate. In a still further aspect, stripping control target molecules are differently labeled compared to other target molecules in a target population.

In another embodiment, performance control features also exist on the array and are complementary to correspond to performance control target molecules which are added to each sample being analyzed. Such performance control features are employed to assess any degradation in the overall performance of the microarray (including, but not limited to signal to noise, dynamic range, linearity of response, and background) attributable to the re-use process. Such features would comprise sequences at pre-defined positions within the array layout that bind, under the employed hybridization conditions, to components of labeled performance control target molecules that possess complementary sequences.

In one embodiment, an array according to the invention is contacted with a plurality of target populations. In one aspect, at least two of the plurality of target populations comprise a least one different sequence. In another embodiment, at least two of the target populations are from two different samples, e.g., from at least two different cell types. In one aspect, at least one sample is from a patient suspected of having a disease (e.g., cancer), phenotype, and/or genotype, while the other sample is from a patient known not to have the disease, phenotype, and/or genotype. In a further aspect, at least one sample comprises biopolymers from cells exposed to an agent, while the other sample comprises biopolymers from cells, which have not been exposed to the agent. In still another aspect, the target populations are from the same type of sample or are different aliquots of one sample, e.g., where an assay is being repeated to replicate and/or validate results. In such cases the target populations may only differ by the presence of different control targets for hybridizing to different patterns of control features on the array. Generally, target populations can be labeled with the same or a different label.

In one aspect, a target population contacted with an array in a given assay comprises at least two sets of target populations, which can be derived from different sample sources. For example, in one aspect, a target population contacted with the array comprises a set of target molecules from a reference sample and from a test sample. In one aspect, the reference sample is from an organism having a known genotype and/or phenotype, while the test sample has an unknown genotype and/or phenotype or a genotype and/or phenotype that is known and is different from that of the reference sample. For example, in one aspect, the reference sample is from a healthy patient while the test sample is from a patient suspected of having cancer or known to have cancer.

In one embodiment, a target population being contacted to an array in a given assay comprises at least two sets of target populations that are differentially labeled (e.g., by spectrally distinguishable labels). In one aspect, control target molecules in a target population are also provided as two sets, e.g., a first set labeled with a first label and a second set labeled with a second label corresponding to first and second labels being used to label reference and test target molecules, respectively.

In one aspect, for example, where the array is being used in a comparative genome hybridization assay, the control target molecules in a population are present at a level comparable to a haploid amount of a gene represented in the target population. In another aspect, the control target molecules are present at a level comparable to a diploid amount of a gene. In still another aspect, the control target molecules are present at a level that is different from a haploid or diploid amount of a gene represented in the target population.

Samples from which target populations of nucleic acids are derived can be obtained from a variety of sources. A sample can include, but is not limited to a sample of tissue, cell(s) or fluid isolated from an individual (animal or plant), including but not limited to: plasma; serum; spinal fluid; semen; lymph fluid; the secretions of skin, respiratory, intestinal, genitourinary tracts; tears; saliva; milk; blood cells; tumors, organs, and also samples of in vitro cell culture constituents (including, but not limited to, conditioned medium resulting from the growth of cells in cell culture medium, putatively virally infected cells, recombinant cells, and cell components), archival samples, fixed samples (e.g., formalin-fixed samples), paraffin-embedded samples, frozen samples, primary tissue samples, cultured samples, samples from embryos (e.g., preimplantation embryos), from amniotic fluid, from chorionic villus tissue, from sperm or oocytes, from laser capture microdisection, biopsies, flow cytometry separations, and the like. In one aspect, the nucleic acid sample is from a mammalian source. In another aspect, the nucleic acid sample is from a mouse, a rat, or a human being.

In one aspect, the nucleic acids comprise genomic nucleic acids such as genomic DNA. A sample of nucleic acids may be prepared using any convenient protocol. In many embodiments, a genomic source is prepared by first obtaining a starting composition of genomic DNA, e.g., a nuclear fraction of a cell lysate, where any convenient means for obtaining such a fraction may be employed and numerous protocols for doing so are well known in the art. The genomic source can comprise genomic DNA representing the entire genome from a particular organism, tissue or cell type or mixture of cell types, developmental stage, and the like.

A given initial genomic source may be prepared from a subject, for example, a plant or an animal that is suspected of being homozygous or heterozygous for a deletion or amplification of a genomic region.

In certain embodiments, the average size of the constituent molecules that make up the initial genomic source typically have an average size of at least about 1 Mb, where a representative range of sizes is from about 50 to about 250 Mb or more, while in other embodiments, the sizes may not exceed about 1 MB, such that the may be about 1 Mb or smaller, e.g., less than about 500 Kb, etc.

Where desired, the initial genomic source may be fragmented, to produced a fragmented genomic source, where the molecules have a desired average size range, e.g., up to about 10 Kb, such as up to about 5 Kb or up to about 1 Kb, where fragmentation may be achieved using any convenient protocol, including but not limited to: mechanical protocols, e.g., sonication, shearing, etc., chemical protocols, e.g., enzyme digestion, etc.

In certain aspects, an initial genomic source is contacted to proteins, such as cellular proteins (in vivo or in vitro). In one aspect, proteins are cross-linked to genomic DNA (e.g., using formaldehyde, UV or other methods known in the art). DNA protein-complexes can be isolated, e.g., by immunoprecipitation or an affinity-based sorting method. In one aspect, DNA is sheared prior to immunoprecipitation. In another aspect, crosslinks are reversed (e.g., by exposing to heat) and the DNA is applied to the array for detection of sequences bound to one or more proteins of interest.

Amplification may or may not occur prior to any fragmentation step.

In one embodiment, an enzyme-based amplification step is used which does not substantially reduce the complexity of the initial genomic source of nucleic acids, e.g., genomic DNA is obtained without a pre-selection step and amplification employs a random set of primers or primers whose complements occur at a desired frequency throughout the genome or whose complements are engineered to be included in a plurality (e.g., all) genomic fragments obtained from a sample (e.g., such as linkers ligated to the ends of genomic fragments). In one aspect, amplification results in an amplified version of virtually the whole genome, if not the whole genome, where the fragmentation, if employed, may be performed pre- or post-amplification.

In certain embodiments, non-reduced complexity nucleic acids are ones in which substantially all, if not all, of the sequences found in the initial genomic source (and organism genome from which the initial source is obtained) are present in the nucleic acid population. By “substantially all” is meant typically at least about 75%, such as at least about 80%, at least about 85%, at least about 90% or more, including at least about 95%, at least about 95% etc, of the total genomic sequences are present in the population, where the above percentage values are number of bases in the population as compared to the total number of bases in the genomic source. Because substantially all, if not all, of the sequences found in the genomic source are present in the sample population of nucleic acids (which can be an amplified population of nucleic acids), the resultant population is not one that is reduced in complexity with respect to the initial genomic template.

Methods for amplifying nucleic sequences using enzymes can vary. In one aspect, genomic nucleic acid is amplified using an isothermal amplification technique. In another aspect, nucleic acid is amplified using a strand displacement technique, such as multiple strand displacement. In a further aspect, the nucleic acid is amplified using random primers, degenerate primers and/or primers which bind to a constant sequence ligated to ends of genomic fragments in a sample.

The primers may be prepared using any suitable method, such as, for example, the known phosphotriester and phosphite triester methods, or automated embodiments thereof. In one such automated embodiment, dialkyl phosphoramidites are used as starting materials and may be synthesized as described by Beaucage et al. (1981), Tetrahedron Letters 22, 1859. One method for synthesizing oligonucleotides on a modified solid support is described in U.S. Pat. No. 4,458,066. Methods for producing random primers are also described in U.S. Pat. Nos. 5,043,272 and 5,106,727, for example. Randomness of in a primer sequence may be introduced by providing a mixture of nucleic acid residues in the reaction mixture at one or more addition steps (to produce a mixture of oligonucleotides with random sequence at that residue position). Thus, an oligonucleotide that is random throughout its length can be generated by sequentially incorporating nucleic acid residues from a mixture of 25% of each of dATP, dCTP, dGTP, and dTTP, to form an oligonucleotide. Other ratios of dNTPs can be used (e.g., more or less of any one dNTP, with the other proportions adapted so the whole amount is 100%).

In one embodiment, multiple displacement amplification (MDA) is used to amplify a genomic sample. In one aspect, the method comprises obtaining a genomic nucleic acid sample and contacting the sample with a phi29-like polymerase using random primers or primers complementary to frequently represented sequences in the nucleic acid sample. In one aspect, polymerase comprises a 3′-5′ exonuclease proofreading activity. In another aspect, the polymerase comprises an error rate which is less than the error rate of Taq polymerase, e.g., an error rate of less than about 1×10⁻⁴, less than about 1×10⁻⁵, less than about 5×10⁻⁶ (in mutations/nucleotide) in the amplified DNA, or less than about 1×10⁻⁶.

In one embodiment, the amount of input genomic DNA is small, comprising less than about 600 ng, less than about 500 ng, less than about 250 ng, less than about 100 ng, less than about 50 ng, less than about 10 ng, less than about 5 ng, or about 1 ng of genomic DNA. In one aspect, the enzyme-based amplification method provides an at least about 1,000-fold amplification of DNA, an at least about 2000-fold amplification, an at least about 5000-fold amplification or an at least about 10,000-fold amplification. In another aspect, genomic DNA is amplified without prior processing steps other than cell lysis and, optionally, dilution, e.g., without centrifugation, addition of chaotropic agents, solvents, alcohols, contacting to a column to remove contaminants and DNA-drying procedures. However, in other aspects, a genomic sample may be contacted to a matrix comprising one or more types of binding molecules for removing undesired sample components. In certain aspects, e.g., where the nucleic acid sample is from an archival source, such as a paraffin-embedded sample or frozen tissue sample, the sample is processed to obtain suitable amounts of template for subsequent a subsequent amplification procedure.

In certain embodiments, samples are processed to reduce the complexity of the sample. In one aspect, a sample comprises substantially a single chromosome, e.g., such as obtained after flow sorting nucleic acids in a sample source. In other embodiments, nucleic acids are sorted to obtain specific categories of nucleic acids, e.g., such as nucleic acids which bind to one or more nucleic acid binding sequences, such samples may comprise genomic DNA, transcribed molecules (e.g., RNA) or copies thereof. In other aspects, modified DNA sequences are selected (e.g., such as methylated sequences).

In certain aspects, the amplified DNA is added directly to subsequent genetic assays without the need for further DNA purification procedures. For example, the amplified and labeled, if necessary, DNA can be applied directly to an array as described further below. In one aspect, the amplified DNA is labeled prior to application to the array.

Nucleic acids may be diluted as necessary. In one aspect, a sample to be amplified comprises from about 10 pg to about 10 ng in 100 μl of an appropriate reaction buffer, e.g., comprising 37 mM Tris-HCl (pH 7.5); 50 mM KCl; 10 mM MgCl₂; 5 mM (NH₄)₂SO₄; 1 mM dATP, dTTP, dCTP, and dGTP; 50 μM exonuclease-resistant primer; 1 unit/mL yeast pyrophosphatase; and 800 units/mL φ29 DNA polymerase, such as described in Hosano, et al., 2003, supra. The nucleic acids are contacted with primers and polymerase under suitable binding conditions to promote binding between the primers and genomic sequences. In one aspect, reactions are incubated at 30° C. for 16 hours and terminated by heating to 65° C. for 3 minutes.

In one embodiment, the enzyme used in the enzyme-based amplification procedure includes a helicase. In one aspect, input DNA from a sample (e.g., genomic DNA or DNA copies of RNA molecules) is contacted with a helicase (e.g., such as E. coli UvrD helicase) for unwinding the input DNA. In certain aspects, helicase is used in conjunction with mutL. Unwound DNA is contacted with single-stranded DNA binding proteins (e.g., such as T4 gene 32 protein (available from Roche Applied Science), and the like), and appropriate concentrations of primers (such as random primers, degenerate sequence primers and the like), and contacted with a DNA polymerase (such as an exonuclease-deficient Klenow fragment of DNA polymerase I) in the presence of dNTPs. Samples are incubated under suitable amplification conditions. In one aspect, isothermal conditions such as incubation at 37° C. are employed throughout the procedure. However, in certain aspects, template is heated at 95° C. to denature and brought to 37° C. in 1-4 minutes, for subsequent amplification for approximately 1-2 hours.

Other whole genome amplification methods may be performed such as DOP (Telenius, Genomics 1992; 13:718-725) and PEP (Zhang, et al., Proc. Natl. Acad. Sci. 1992; 89:5847-5851). In one aspect, an amplification method is selected which provides a minimal amount of bias.

However, in certain other embodiments, nucleic acids from a sample are directly labeled (e.g., by random priming using Klenow) and applied directly to the array, e.g., without an enzyme-based amplification step.

In still a further aspect, signal from nucleic acid targets bound to an array are amplified using a signal-based amplification, e.g., such as a bDNA-based amplification method.

In one aspect, in generating labeled nucleic acids for application to an array, a nucleic acid template (e.g., genomic DNA, RNA, cRNA, cDNA, etc.) and a random primer population are employed together in a primer extension reaction that produces labeled nucleic acids. Primer extension reactions for generating labeled nucleic acids are well known to those of skill in the art, and any convenient protocol may be employed. Primers are contacted with a template in the presence of a sufficient polymerase under primer extension conditions sufficient to produce the desired primer extension molecules. Polymerases of interest include, but are not limited to, polymerases derived from E. coli, thermophilic bacteria, archaebacteria, phage, yeasts, Neurosporas, nemotodes, Drosophila sp, primates and rodents, as well as polymerases derived synthetically from several species or by in silico modeling. The polymerase extends the primer according to the template to which it is hybridized in the presence of additional reagents which may include, but are not limited to: dNTPs; monovalent and divalent cations (e.g. KCl, MgCl₂); sulfhydryl reagents (e.g. dithiothreitol); and buffering agents, e.g. Tris-HCl.

In one aspect, the reagents employed in the subject primer extension reactions include a labeling reagent, where the labeling reagent may be the primer or a labeled nucleotide, which may be labeled with a directly or indirectly detectable label. A directly detectable label is one that can be directly detected without the use of additional reagents, while an indirectly detectable label is one that is detectable by employing one or more additional reagents, e.g., where the label is a member of a signal producing system made up of two or more components. In many embodiments, the label is a directly detectable label, such as a fluorescent label, where the labeling reagent employed in such embodiments is a fluorescently tagged nucleotide(s), e.g., dCTP. Fluorescent moieties which may be used to tag nucleotides for producing labeled probe nucleic acids include, but are not limited to: fluorescein, the cyanine dyes, such as Cy3, Cy5, Alexa 555, Bodipy 630/650, and the like. Other labels may also be employed as are known in the art.

In one aspect, the labeling procedure does not alter the complexity of the sample to any significant extent as compared to the initial unlabeled source (e.g., such as the initial genomic source). A number of different nucleic acid labeling protocols are known in the art and may be employed to produce a population of labeled probe nucleic acids. The particular protocol may include the use of labeled primers, labeled nucleotides, modified nucleotides that can be conjugated with different dyes, a non-amplifying primer extension protocol (e.g., a single product is produced per template strand), one or more amplification steps, etc.

In certain embodiments, an array is contacted with a nucleic acid sample under stringent assay conditions, i.e., conditions that are compatible with producing bound pairs of biopolymers of sufficient affinity to provide for the desired level of specificity in the assay while being less compatible to the formation of binding pairs between binding members of insufficient affinity. Stringent assay conditions are the summation or combination (totality) of both binding conditions and wash conditions for removing unbound molecules from the array.

As known in the art, “stringent hybridization conditions” and “stringent hybridization wash conditions” in the context of nucleic acid hybridization are sequence dependent, and are different under different experimental parameters. Stringent hybridization conditions include, but are not limited to, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization in 0.5 M NaHPO4, 7% sodium dodecyl sulfate (SDS), 1 mM EDTA at 65° C., and washing in 0.1×SSC/0.1% SDS at 68° C. can be performed. Additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

Wash conditions used to remove unbound nucleic acids may include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature. Other methods of agitation can be used, e.g., shaking, spinning, and the like.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate. The term “highly stringent hybridization conditions” as used herein refers to conditions that are compatible to produce complexes between complementary binding members, i.e., between immobilized probes and complementary sample nucleic acids, but which does not result in any substantial complex formation between non-complementary nucleic acids (e.g., any complex formation which cannot be detected by normalizing against background signals to interfeature areas and/or control regions on the array).

Additional hybridization methods are described in references describing CGH techniques (Kallioniemi et al., Science 1992; 258:818-821 and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol. 1981; 21:470-480 and Angerer et al., In Genetic Engineering: Principles and Methods, Setlow and Hollaender, Eds. Vol 7, pgs 43-65 (Plenum Press, New York 1985). See also U.S. Pat. Nos. 6,335,167; 6,197,501; 5,830,645; and 5,665,549; the disclosures of which are herein incorporated by reference.

In one embodiment, after contacting with a first population of nucleic acids and prior to contacting with a subsequent population, an array is exposed to stripping conditions for removing bound target molecules from the array. In certain aspects, exposing the array to stripping conditions comprises altering one or more of: temperature, pH conditions, and types and concentrations of denaturants. For example, in one aspect, the array is exposed to a temperature of greater than about 60° C., 70° C., greater than about 80° C., greater than about 90° C., greater than about 95° C., or about 100° C. or greater.

In one embodiment, an array is exposed to a low salt concentration solution (e.g., comprising <50 μM salt). In still another embodiment, the array is exposed to a to a denaturant, such as urea (e.g., >2 M) or formamide In a further aspect, the array is exposed to conditions that degrade target molecules while leaving probe molecules substantially intact, such as by exposing the array to alkaline conditions (e.g., by contacting the array with a solution comprising >5 mM NaOH) and/or by exposing the array to nucleases (e.g., RNAses) that degrade target molecules but not probe molecules. Combinations of the above conditions can be used. For example, the array can be exposed to a low salt solution comprising a denaturant and/or exposed to conditions that degrade target molecules while leaving probe molecules substantially intact.

The amount of time during which an array is exposed to the stripping condition can vary and in one aspect varies inversely with temperature, e.g., stripping time can be decreased by increasing temperatures. In one aspect, an array is exposed to stripping solution (e.g., such as a low salt solution and/or solution comprising denaturant) a plurality of times (e.g., about 2 to about 5 times or more). Reagents for enhancing the stability of probes on the array while targets are being stripped can be added, e.g., in certain aspects, the stripping solution is substantially nuclease-free and/or detergents are added (e.g., such as 0.1-1% SDS).

Stripping conditions will vary with the type of biopolymer used to form features on the array. While conditions for nucleic acid arrays are disclosed above, in certain embodiments the invention further relates to the stripping and reuse of polypeptide arrays. In one aspect, stripping conditions include contacting an array comprising bound target polypeptides and detecting the presence of a pattern of control polypeptide probes (a first member of a binding pair, e.g., such as an antibody, antigen-binding fragment, affibody, aptamer, and the like) which bind to control target molecules (a second member of a binding pair which specifically binds to the first member of the binding pair) to determine the efficacy of stripping. In one aspect, stripping conditions include, for example, contacting the array with about 1-10% SDS, e.g., about 2% SDS. Additonal detergents may also be used, e.g., such as 0.05 to about 5% Tween or about 1% Tween.

In another aspect, a stripping solution comprises a 1-10% SDS solution, 10-150 mM β-mercaptoethanol, and Tris-HCl buffer, at a pH of about 6-7.0. Stripping can be performed at temperatures from about 25° C. to about 100° C., or from about 50° C. to about 100° C. depending on pH and other conditions during the stripping process. In another aspect, a stripping solution comprises a low pH buffer (e.g., 0.2M glycine, pH2.2). In a further aspect, a stripping solution comprises a denaturant, e.g., such as ammonium sulfate (e.g., 1M) and/or urea (e.g., 1M).

The array may be directly contacted with the second target population; however in one aspect, the array is dried after exposing to stripping conditions, e.g., by air drying or exposing to a nitrogen gun or by moving the array to a drying chamber for exposing to controlled drying conditions.

Detection of a pattern of complexes formed by stripping control feature probes binding to stripping control targets spiked into a given population of targets can provide a means of evaluating the efficacy of a stripping procedure. For example, in one aspect, detection of signal corresponding to a first pattern of complexes formed in a first assay after stripping and contacting to a target population comprising second stripping control target molecules provides an indication that stripping did not completely remove complexes from the first assay.

A number of different responses may follow. For example, in one aspect, when the signal corresponding to the first pattern exceeds a threshold signal, data from the second assay may be discarded, or associated with a data flag, e.g., by a person, visually inspecting an image of signals corresponding to binding to the array, or by a processor of an instrument that is calibrated to detect signals that deviate from a predetermined threshold signal. The data flag can be used to provide an indication that the data may represent cumulative binding of targets from both assays. The data flag may serve as a trigger to indicate that data from the second assay should be normalized in a way that accounts for residual binding from the first assay.

In still other aspects, for example, where performance control target molecules are added to each sample, a data flag may be associated with signal obtained from complexes formed between performance control probe molecules and performance control targets when the relative ratios of signals corresponding to different performance control target molecules are different from the ratios spiked in to the sample. Where this difference exceeds a predetermined threshold, the date flag may serve to indicate that data from the performance controls indicate unacceptable degradation in the overall performance of the microarray (including, but not limited to signal to noise, dynamic range, linearity of response, and background) attributable to the re-use process. However, in another aspect, a data flag indicates the data should be discarded or at least not used to derive any conclusions.

In another aspect, a data flag may provide a trigger to calibrate an array scanner or other instrument being used to identify binding complexes between targets and probes on the array. For example, the array scanner or instrument may be calibrated to detect only those signals whose intensity or value are greater than a signal associated with the first pattern after stripping and/or after exposing to the second target population.

In still other aspects, signal corresponding to the first pattern is detected after stripping but prior to contacting with a second population of target molecules. Where the signal exceeds a predetermined threshold value, the array may be re-exposed to additional stripping procedures which are the same or different from the initial stripping procedure.

Reading the resultant hybridized array may be accomplished by illuminating the array and reading the location and intensity of signals associated with a probe:taget complex, such as resulting fluorescence, at each feature of the array to detect any binding complexes on the surface of the array. For example, a scanner may be used for this purpose, which is similar to the AGILENT MICROARRAY SCANNER available from Agilent Technologies, Palo Alto, Calif. Other suitable devices and methods are described in U.S. patent application Ser. No. 09/846,125 “Reading Multi-Featured Arrays” by Dorsel et al.; and U.S. Pat. No. 6,406,849. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of nucleic acids, and are suitable for some embodiments.

Results from the reading or evaluating may be raw results (such as fluorescence intensity readings for each feature in one or more color channels) or may be processed results (such as those obtained by subtracting a background measurement, or by rejecting a reading for a feature which is below a predetermined threshold, normalizing the results, and/or forming conclusions based on the pattern read from the array (such as whether or not a particular target sequence may have been present in the sample, or whether or not a pattern indicates a particular condition of an organism from which the sample came).

In one aspect, the level of binding of labeled target molecules to probe molecules at a feature is obtained by measuring the surface density of the bound label (or of a signal resulting from the label).

In certain embodiments, binding to a probe molecule may be assessed by evaluating its binding to two populations of nucleic acids that are distinguishably labeled. In these embodiments, for a single probe of interest, the results obtained from hybridization with a first population of labeled nucleic acids may be compared to results obtained from hybridization with the second population of nucleic acids, usually after normalization of the data. The results may be expressed using any convenient means, e.g., as a number or numerical ratio, etc.

In certain embodiments, the methods include a step of transmitting data from at least one of the detecting and deriving steps, as described above, to a remote location. The data may be transmitted to the remote location for further evaluation and/or use. Any convenient telecommunications means may be employed for transmitting the data, e.g., facsimile, modem, internet, etc.

In still other aspects, the ratio of signal attributable to stripping control probe: stripping control target complexes having a first label to stripping control probe: stripping control target complexes having a second label is determined as a metric to evaluate whether stripping effects both labeled populations of target molecules equally. In one aspect, where stripping control probe features are bounded by negative stripping control features, stripping is considered effective when signal remaining at the stripping control features does not significantly differ in intensity from the mean signal at the negative stripping control features. In another aspect, stripping is considered effective when signal remaining the stripping control features does not significantly differ in intensity from the mean signal observed at interfeature areas. In still other aspects, although a significant amount of signal at the stripping control features is observed, stripping is still considered effective at a predetermined threshold or setpoint to which a scanner may be calibrated to detect only signal intensities higher than the threshold. For example, in one aspect, the threshold is lower than a signal expected for sequence present in a haploid amount in a target population of nucleic acids to be assayed or for a gene that is expressed at single copy per cell level of a gene copy or transcript for a low abundance gene in a target population.

In certain aspects, one or more of: binding to form binding complexes between target and probe molecules, washing to remove unbound targets, scanning to detect complexes, and stripping to remove bound targets, can be automated, e.g., using a device comprising reaction chambers for contacting the array with desired fluids (e.g., liquids and/or gases) at controlled temperatures and times.

In one aspect, an array is moved from a chamber comprising a binding solution (e.g., a population of target molecules, including control target molecules) to a chamber comprising a washing solution for removing unbound target molecules to a chamber for scanning the array, to a stripping chamber for removing bound target molecules, to a chamber comprising a new binding solution (e.g., comprising a different population of target molecules). The steps can be repeated multiple times. One or more steps of the process can be performed in a single reaction chamber or each can be performed in a different chambers. In one aspect, binding and washing occurs in a chamber that can be placed in a scanner. In another aspect, binding, washing and stripping (not necessarily in that order) occurs in a chamber that can be placed in a scanner, e.g., the chamber can comprise an at least partially transparent surface for transmitting emitted light form the array to the scanner. Chambers can be integrated in a single instrument or in a plurality of instruments. In certain aspects, a reaction chamber comprises a drain for removing fluids from the chamber. In still other aspects, a reaction chamber comprises an inlet for introducing fluids into a chamber (e.g., such as a binding, washing or stripping solution or a gas for drying an array within the chamber. In still other aspects a plurality of inlets may be provided which communicate independently with the chamber (e.g., through the use of valves or pumps). The inlets may introduce fluids into the chamber via multiple inlet ports or via a single entry conduit, which a plurality of inlets join. Fluid flow through the entry conduit can be independently controlled via means of valves or pumps as discussed above.

Fluid flow through one or more chambers and conditions in one or more chambers (e.g., temperature, pH, etc.) can be controlled by a processor in operable communication with the one or more chambers. In one aspect, stripping conditions can be controlled by the processor based on the type of assay being conducted. For example, stripping conditions may be different for a CGH assay, location analysis assay or gene expression assay. In one aspect, for example, in a gene expression assay, a target population may include RNA molecules, which can be removed from the array by enzymatic digestion and/or exposure to alkaline conditions. In another aspect, e.g., in a CGH assay, or genomic location analysis assay, stripping conditions may comprise one or more of the use of high temperatures, denaturants and the like. In one aspect, the processor receives input from a scanner relating to signal intensity at stripping control features after a stripping procedure and implements a protocol for re-exposing to stripping conditions based on the signal intensity at the stripping control features.

In still another embodiment, an array is associated with an identifier such as a bar code or RFID tag, and a device comprising the one or more chambers comprises a fixed identifier reader or one which is removed from, or removable from, a device comprising the chamber. For example, the device may include a fixed barcode reader or hand-held bar code reader that can be affixed to, and removable from, the device or placed at a station in proximity to the device. The identifier can be associated with data relating to the array, including, but not limited to: the nature of probes on the array, a pattern in which one or more control probe features is disposed, the nature of a procedure or condition to which the array has been or should be exposed, and the like. In one embodiment, the identifier is associated with data relating to one or more of: binding conditions to which the array has been exposed, washing conditions to which the array has been exposed, stripping conditions to which the array has been exposed, and the like. In one aspect, information associated with the identifier can be updated or a new identifier can be added. For example, an array removed from the device and scanned or scanned within the device (where a scanner is part of the device) may be associated with data relating to new conditions to which the array should be exposed (e.g., such as additional stripping and/or washing conditions, additional binding reactions, etc.). The updated data/or new identifier may be read by the device or an identifier reader associated with the device and instructions for executing new and/or additional procedures may be provided to the device by a processor in communication with both the identifier reader and the device. In one aspect, the identifier is remotely programmable and/or data associated with the identifier is updated remotely, and an array need not be removed from the device in order to execute the new and/or additional procedures. In certain aspects, when a user selects a procedure (e.g., hybridization, washing, stripping, etc) to be executed by the device or alters parameters of a procedure, information relating to that procedure and/or parameters is automatically stored in a memory accessible by the processor and associated with the identifier on the array. In summary, the identifier provides the means to track the history and performance of processing of an array associated with a particular experimental sample.

In a further embodiment, the invention provides computer program products comprising computer readable media with instructions for automating one or more of the operations above. For example, computer program products according to the invention can comprise instructions for calibrating a scanner based on inputted data relating to a signal from a stripping control pattern and/or performance control features and can include further instructions for comparing the signal to a predetermined threshold. In certain aspects, the predetermined threshold is unique to a particular subsequent assay being performed, e.g., whether the assay is a gene expression assay, CGH assay, or location analysis assay, and program may include instructions for obtaining input relating to a particular assay type and setting a threshold based on the assay type. In certain aspects, the predetermined threshold with respect to stripping controls is a signal which is less than about 5-fold, less than about 4-fold, less than about 3-fold, less than about 2-fold, less than about 1.5-fold, less than about 1-fold, or less than about 0.5 fold. different from a background signal (e.g., from an interfeature or inter-array area) on an array substrate in a particular assay employed (e.g., a gene expression assay, CGH assay or location analysis assay. Similarly, computer program products can comprise instructions for executing operations of a reaction chamber in which an array is being stripped based on inputted data relating to a control pattern and/or assay in which the array is going to be reused.

In still a further aspect, computer program products receive inputs relating to one or more of stripping conditions, hybridization conditions, washing conditions, and the like and associates the conditions with value(s) for signals associated with stripping control probe: stripping control target complexes and, optionally, performance control probe:performance control target complexes, obtained after stripping to identify quality control metrics to be associated with a particular stripping condition. In another aspect, the computer program product is associated with a memory which comprises data associating an identifier on an array with a protocol to which the array has been or should be exposed,

Arrays according to the invention and stripping control target molecules, and optionally, performance control target molecules, can be used in a variety of assays, where it may be desirable to use a single array a plurality of times. In one aspect, an array according to the invention is used in a gene expression assay, e.g., to detect patterns of expression associated with a disease state, a physiological response, a developmental stage, exposure to an agent or environmental condition, exposure to a drug, and the like. For example, in one aspect, a population of target nucleic acids, representing transcription products (e.g., RNA transcripts, cRNA, or cDNA) (which may be labeled) and further including stripping control target molecules, and optionally, performance control target molecules, is contacted with a population of probe nucleic acids under hybridization conditions. In certain aspects, the population of target molecules can comprise a set of two different types of target molecules (e.g., from different sample types, such as different cell populations, from the same type of cell population exposed to different conditions or agents, and the like), and members of each set may be distinguishable by differential labeling. Following hybridization, non-bound target is removed or separated from the probe, e.g., by washing. Washing results in a pattern of hybridized target, which may be read using any convenient protocol, e.g., with a fluorescent scanner device where fluorescent labels are employed. From this pattern, information regarding the mRNA expression profile in the initial mRNA sample from which the target population was produced may be readily derived or deduced.

In certain aspects, the array provides probes for screening or scanning a genome of an organism and comprises probes from a plurality of regions of the genome. In one aspect, the array comprises probe sequences for scanning an entire chromosome arm, wherein probes targets are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In another aspect, the array comprises probes sequences for scanning an entire chromosome, a set of chromosomes, or the complete complement of chromosomes forming the organism's genome. In certain aspects, the array comprises both non-coding and coding sequences. In one aspect, individual probes on the array comprise either coding or non-coding sequences but do not comprise both coding and non-coding sequences. In certain aspects, individual probes comprise sequences that do not normally occur together, e.g., to detect gene rearrangements, for example.

For example, in one embodiment, arrays according to the invention are used in a comparative genome hybridization (CGH) assay. In one aspect, the target population comprises at least two sets of nucleic acids, one from a test sample and one from a reference sample. As discussed previously, nucleic acid samples can be obtained from a variety of sample sources. In one aspect, a test sample is from a biopsy. In another aspect, the test sample is from a tumor. In still another aspect, the sample is from a source of fetal nucleic acids, such as amniotic fluid or chorionic villus cells. The source of reference nucleic acid also can vary. In one aspect, the reference nucleic acid is from a healthy patient. In another aspect, the reference nucleic acid is from an individual known to have a diploid complement of a nucleic acid or a known amount of the nucleic acid. Thus, in certain aspects, the reference nucleic acid is from the test sample. In one aspect, the reference sample includes one or more paralogous sequences that correspond to test sequences whose copy number is being evaluated.

A given initial genomic source may be prepared from a subject, for example a plant or an animal, which is suspected of being homozygous or heterozygous for a deletion or amplification of a genomic region. In certain embodiments, the average size of the constituent molecules that make up the initial genomic source typically have an average size of at least about 1 Mb, where a representative range of sizes is from about 50 to about 250 Mb or more, while in other embodiments, the sizes may not exceed about 1 MB, such that the may be about 1 Mb or smaller, e.g., less than about 500 Kb, etc. Where desired, the initial genomic source may be fragmented as desired, to produce a fragmented genomic source, where the molecules have a desired average size range, e.g., up to about 10 Kb, such as up to about 1 Kb, where fragmentation may be achieved using any convenient protocol, including but not limited to: mechanical protocols, e.g., sonication, shearing, etc., chemical protocols, e.g., enzyme digestion, etc.

As discussed above, the initial genomic source may be amplified by an enzyme-based amplification procedure, where the amplification may or may not occur prior to any fragmentation step. In those embodiments where the prepared nucleic acid has substantially the same complexity as the initial genomic source from which it is prepared, the amplification step employed is one that does not reduce the complexity, e.g., one that employs a set of random primers, degenerate primers, ligated primers comprising constant regions, primers hybridizing to repetitive regions, and the like. For example, the initial genomic source may first be amplified in a manner that results in an amplified version of virtually the whole genome, if not the whole genome, before labeling, where the fragmentation, if employed, may be performed pre- or post-amplification.

As discussed above, in one aspect, the prepared collection of nucleic acids to be applied to an array is a “non-reduced-complexity” collection of nucleic acids, as compared to the initial genomic source and genome of the organism from which the initial genomic source is obtained. A non-reduced complexity collection is one that is not produced in a manner designed to reduce the complexity of the sample, e.g., is not produced using collections of primers that are designed to prime only a certain percentage or fraction of the initial genomic source. In contrast, a reduced complexity collection of nucleic acids is one that has been produced by a protocol that only amplifies a certain portion, fraction or region of the genomic source used to prepare the collection or is selected (e.g., by a sorting procedure) to remove certain classes of nucleic acids from a sample (e.g., such as nucleic acids which do not bind to a binding protein, as discussed further below or flow-sorted chromosomes, and the like).

In certain embodiments, non-reduced complexity collections of nucleic acids are ones in which substantially all, if not all, of the sequences found in the initial genomic source (and organism genome from which the initial source is obtained) are present in the prepared population of nucleic acids being applied to the array. By substantially all is meant typically at least about 75%, such as at least about 80%, at least about 85%, at least about 90% or more, including at least about 95%, at least about 95% etc, of the total genomic sequences are present in the prepared population to be applied to the array, where the above percentage values are number of bases in the prepared population as compared to the total number of bases in the genomic source. Because substantially all, if not all, of the sequences found in the genomic source are present in the prepared population of nucleic acids, the resultant population of nucleic acids is not one that is reduced in complexity with respect to the initial genomic template, i.e., it is not a reduced complexity population of nucleic acids.

A non-reduced complexity collection of prepared nucleic acids can be identified or validated by screening the collection using a genome wide array of probe nucleic acids for the genomic source of interest. Thus, one can tell whether a given collection of nucleic acids has non-reduced complexity with respect to its genomic source by assaying the collection with a genome wide array for the genomic source. The genome wide array of the genomic source is an array of probe nucleic acids in which the entire genomic source is screened at a sufficiently high resolution, where the resolution is typically at least about 1 Mb, e.g., at least about 500 Kb, such as at least about 250 Kb, including at least about 100 Kb, e.g., 50 Kb or higher (such as 25 Kb, 15 Kb, 10 Kb or higher), where resolution in this context means lengths of the genomic source between regions present on the array in the form of immobilized probes. In such a genomic wide assay of sample, a non-reduced complexity sample is one in which substantially all of the array features on the array provide a positive signal, where by substantially all is meant at least about 50%, such as at least about 60, 70, 75, 80, 85, 90 or 95% (by number) or more.

The ratio of binding of target molecules in the test sample to target molecules from the reference sample can be used to detect relative copy number of nucleic acids in the two samples. In certain assays, the target population can comprise a single type of sample, e.g., such as test sample, while another type of sample, such as a reference sample, is applied to a second array, which comprises the same probe molecules. In one aspect, the same stripping control molecules are spiked into the reference sample for application to the second array as are included in the test sample.

In one embodiment, the ratio of complexes formed by test molecules and complexes formed by reference molecules on one or more arrays is used to determine the copy number of a nucleic acid in a sample (such as a genomic DNA sample). Copy number determination can include the determination of both gain and loss of sequences. In a one aspect, copy number determination is correlated with one or more characteristics of a patient supplying the sample, e.g., such as a disease afflicting the patient or the risk for a patient of developing disease symptoms. In another aspect, copy number determination is associated with changes in characteristics of a patient, e.g., by comparing samples of nucleic acids from a patient obtained at different time periods. Copy number determinations can be used for prenatal testing of chromosomal imbalances (e.g., such as trisomy 21) and both germline and somatic cells can be sample sources for methods of the invention.

In still another aspect, copy number determination is used to screen samples to identify patients at risk for a disease associated with a genomic imbalance such as cancer. In still another aspect, copy number determination is used to screen samples to determine stage and prognosis of a disease associated with a chromosomal imbalance such as cancer. Additional applications are further described in U.S. Pat. Nos. 6,335,167; 6,197,501; 5,830,645; and 5,665,549; the disclosures of which are herein incorporated by reference.

Test and reference nucleic acids (including control target molecules) applied to an array may or may not be labeled, depending on the particular detection protocol employed in a given assay. For example, in certain embodiments, binding events on the surface of a substrate may be detected by means other than by detection of a labeled probe nucleic acids, such as by change in conformation of a conformationally labeled immobilized probe, detection of electrical signals caused by binding events on the substrate surface, etc. In certain embodiments, however, the populations of target nucleic acids are labeled (e.g., test and reference nucleic acids from test and reference samples sources, respectively), where the populations may be labeled with the same label or different labels, depending on the actual assay protocol employed. For example, where each population is to be contacted with different but identical arrays, each target nucleic acid population or collection may be labeled with the same label. Alternatively, where both populations are to be simultaneously contacted with a single array of probes, i.e., cohybridized to the same array of immobilized probe nucleic acids, the populations are generally distinguishably or differentially labeled with respect to each other. In some aspects, there may be more than one test nucleic acid sample and more than one reference nucleic acid sample.

In one aspect, the complexity of a target population of nucleic acids applied to the array is reduced compared to a cell from which the nucleic acids are obtained. In one aspect, complexity of the target is reduced by selecting for particular chromosomes, e.g., by flow sorting. In further embodiments, complexity of the target is reduced by eliminating non-repetitive sequences, e.g., by denaturing a nucleic acid sample, and separating fractions according to their kinetics of reassociation. In still further embodiments, reduction in complexity is achieved through an enzyme-based amplification procedure being used, e.g., by contacting sample nucleic acids with primers whose complements are present in the sample at a lower frequency than the complements of primers used to prepare a non-reduced complexity sample.

In one aspect, a reduced complexity sample is one in which the complexity of nucleic acids in the sample is at least about 20-fold less, such as at least about 25-fold less, at least about 50-fold less, at least about 75-fold less, at least about 90-fold less, at least about 95-fold less, than the complexity of the initial source, in terms of total numbers of sequences found in the prepared sample to be applied to the array as compared to the initial source, up to an including a single gene locus being represented in the collection.

In still other aspects, target nucleic acid sequences are reduced in complexity by selecting nucleic acids based on their ability to bind to one or more nucleic acid binding proteins (e.g., proteins which bind to origins of replication, recombination hotspots, promoters, enhancers, methylation sites, centromeres, telomeres, untranslated regions, introns or exon/intron boundaries including intronic sequences, and the like). In certain aspects, the sequences that bind to the one or more nucleic acid binding proteins are then amplified using a non-biased amplification method (e.g., such as MDA) and applied to the array. However, in other aspects, the sequences are applied to the array without a previous amplification step and optionally, after a direct labeling step. Complexes formed between the amplified sequences and probes on the array are used to identify regions of a genome or transcriptome bound by the nucleic acid binding proteins and/or to identify motif sequences common to these regions. This procedure is referred to herein as “location analysis.”

In certain aspects, nucleic acid sequences bound to nucleic acid binding proteins are cross-linked to their binding proteins (e.g., using formaldehyde or UV treatment) and cleaved with a cleavage agent (such as an exonuclease) or subjected to shearing conditions to remove or decrease the amount of nucleic acid sequences outside of the binding region to which the proteins are bound. See, e.g., as described in U.S. Pat. No. 6,410,243, the entirety of which is incorporated by reference herein. Bound (and optionally crosslinked complexes) can be removed from non-bound nucleic acids (and optionally amplified), thereby enriching the target population for those nucleic acids bound to one or more nucleic acid binding proteins of interest. Separation techniques include immunoprecipitation, or other sorting techniques based on affinity of a binding molecule (e.g., an antibody, a tagged antigen binding protein, an affibody) for the nucleic acid binding protein(s) which can be used to separate bound from unbound molecules. In certain aspects, bound proteins are then removed from the nucleic acids. For example, formaldehyde-generated crosslinks can be reversed by heating. The enriched nucleic acids, representing genome or transcriptome sequences that are bound by the one or more nucleic acid binding proteins of interest are then bound to an array according to the invention. In one aspect, this target population of nucleic acids includes stripping control target nucleic acids which bind to a pattern of stripping control features on the array as described above. In another aspect, the target population further includes performance control target molecules which bind to performance control features, which may or may not be disposed in a pattern on the array.

In certain aspects, a target population comprises a set of target populations that can be differentially labeled, such as a test target population prepared as described above and a reference target population. In one aspect, the reference sample is from a cell which lacks epitopes of proteins of interest bound to nucleic acids in the test sample (such as a cell which lacks the gene for the protein of interest or does not translate the protein of interest), but which is otherwise isogenic to the test cell. In other aspects, the reference sample is a sample that is treated the same as the test sample with the exception that the binding molecule (e.g., such as an antibody) is omitted. The reference population may also include spiked in stripping control target molecules and, optionally, performance control molecules.

In one aspect, an array used in a gene expression assay, a CGH assay, a location analysis assay, or any other array-based assay (e.g., a genotyping assay, screening assay, and the like) can be exposed to stripping conditions and detection of a pattern of complexes formed between stripping control probe features and stripping control probe targets can be used as an indication of the efficacy of the stripping process as described above. As discussed above, the ratio of complexes formed between different performance control probes and performance control target molecules can be compared to the ratio of performance control target molecules in the initial sample, to further assess how the overall performance of the array is affected by the stripping conditions. In certain aspects, the presence of the pattern of complexes is detected prior to proceeding with a subsequent hybridization assay (e.g., to detect gene expression or other properties of a target population, such as copy number of nucleic acids or locations of sequences to which regulatory proteins bind).

As described above, an array can be exposed to a stripping condition after binding to target molecules and detection of bound complexes. The efficacy of the stripping condition can be monitored by detecting signal produced by control targets bound to control features in a pattern on the array. In certain aspects, the pattern is detected (e.g., by rescanning the array after stripping) prior to contacting the array with another population of target molecules. In such cases, signal associated with the pattern that exceeds a predetermined threshold can be used as a trigger to re-expose the array one or more times to the same or different stripping conditions or in some, cases to discard the array. Alternatively, or additionally, an array scanner detecting signals in a subsequent assay may be calibrated to read only those signals that are higher in intensity than the signal associated with the control pattern from a previous assay or one may employ additional performance control molecules as discussed above which enable assessment of actual performance of the microarray in terms such as signal to noise, linearity, dynamic range or background.

However, in another aspect, the pattern is detected after contacting with a new population of target molecules. As described above, detection of signal associated with the pattern which exceeds a predetermined threshold may be used to associate a data flag with the array, such that, e.g., data obtained in the second assay is discarded or discounted or normalized to account for residual binding from the first assay. Alternatively, or additionally, an array scanner may be calibrated to read only those signals that are higher in intensity than the signal associated with the pattern, i.e., after the second assay, the array may be scanned twice, a first time to detect a control pattern from a previous assay, and a second time after an appropriate calibration has been made.

In certain embodiments, a stripped array is used in an assay of the same type as a previous assay. For example, an array used in a gene expression assay may be used in a subsequent gene expression assay; an array used in a CGH assay is reused in another CGH assay and an array used in a location analysis is used in another location analysis. In other embodiments, a stripped array is used in a subsequent assay of a different type. For example, an array used in a gene expression assay can be reused in a CGH or location analysis assay. An array used in a CGH assay can be reused in a gene expression assay or location analysis assay. An array used in a location analysis assay can be reused in a CGH or gene expression assay. Multiple other permutations are possible and arrays may be used 1, 2, 3, 4 or more times in the same and/or different types of assays. While in certain aspects, as discussed above, target molecules applied in subsequent assays are different from those applied in prior assays (e.g., target are from different samples), in certain aspects, the targets are substantially the same, except for the presence of a different control molecule, e.g., where the subsequent assay is used to validate or repeat the results of a previous assay.

Kits for use in the assays described above are also provided. In one aspect, the kits at least include the arrays of the invention. In one embodiment, a kit comprises first and second stripping control target molecules for binding to first and second stripping control features on an array. In another aspect, the kit comprises a plurality of performance control target molecules, wherein members of the set are present in known ratios of concentrations or can be added to a sample at known ratios of concentrations. In one aspect, the sequences of the stripping control target molecules and/or performance control molecules are not otherwise expected to be in a target population of molecules. In one aspect, a performance control target molecules comprise a set of 2 or more, 4 or more, 6 or more, 8 or more, 10 or more sequences, about 24 or more, about 36 or more, about 48 or more or about 96 or a multiple thereof, e.g., 384 or more different probes. Generally, stripping control targets and performance control target molecules contain sequences that are not substantially complementary to experimental sequences being identified or tested. In one aspect, control performance target sequences and experimentally-target sequences (e.g., such as mRNAs or copies thereof) can be labeled with the same types of labels under conditions that permit equivalent labeling efficiencies.

In one aspect, the kit further comprises one or more reagents for exposing an array to a stripping condition, e.g., such as a low salt buffer, a denaturant, a solution of suitable pH for stripping, an agent for preferentially removing target molecules from an array while leaving probe molecules substantially intact, and the like. The kits may further include one or more additional components necessary for carrying out an array-based assay, such as a gene expression assay, CGH assay, location analysis assay, genotyping assay, screening assay, and the like. Such components may include, but are not limited to sample preparation reagents, buffers, labels, and the like. As such, the kits may include one or more containers such as vials or bottles, with each container containing a separate component for the assay, and reagents for carrying out an array assay such as a nucleic acid hybridization assay or the like.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method comprising,
detecting binding of a first population of target molecules to an array;
exposing the array to conditions for stripping bound target molecules from the array;
detecting binding of a first pattern of binding on the array indicating to what extent stripping of bound targets has occurred.

2. The method of claim 1, wherein detecting occurs before or after a second population of target molecules is contacted to the array.

3. The method of claim 1, wherein the first pattern comprises a pattern of binding of first stripping control target molecules present in the first population of target molecules on the array.

4. The method of claim 3, wherein the first pattern comprises a disposition of first stripping control features on the array bound to the first control target molecules.

5. The method of claim 4, wherein the first stripping control features are arranged to form a symbol.

6. The method of claim 5, wherein the symbol is a number or a letter.

7. The method of claim 2, wherein the step of detecting a pattern comprises determining the amount of binding of the first stripping control target molecules.

8. The method of claim 6, wherein the amount of binding of the first stripping control target molecules is compared to a threshold amount.

9. The method of claim 1, wherein when a difference is observed between a threshold amount and the amount of binding of the first stripping control target molecule, the array is not contacted with an additional target population.

10. The method of claim 1, wherein when a difference is observed between a threshold amount and the amount of binding of the first stripping control target, the array is re-exposed to same or different stripping conditions.

11. The method of claim 7, wherein when a difference is observed between the threshold amount and the amount of binding of the first stripping control target, binding data of the second population of molecules is associated with a data flag.

12. The method of claim 2, wherein the second population of target molecules comprises second stripping control target molecules which specifically bind to second stripping control features on the array.

13. The method of claim 12, wherein the second stripping control features are arranged in a second pattern different from the first pattern produced by the disposition of the first stripping control features.

14. The method of claim 1, further comprising detecting target molecules binding to the array prior to stripping.

15. The method of claim 14, wherein the detecting provides data relating to the expression of genes in a sample from which the target molecules are derived.

16. The method of claim 14, wherein the detecting provides data relating to the copy number of nucleic acids in the sample from which the target molecules are derived.

17. The method of claim 14, wherein the detecting provides data relating to the location of sequences to which a nucleic acid binding protein in the sample from which the target molecules are derived, binds.

18. The method of claim 14, wherein the detecting provides data relating to a genotype of a sequence in the target population.

19. The method of claim 2, further comprising detecting target molecules from the second population of target molecules that are bound to the array.

20. The method of claim 13 further comprising detecting the second pattern.

21. The method of claim 1, further comprising calibrating an array scanner to detect signals of higher intensity than the signal associated with the first pattern after stripping.

22. The method of claim 20, further comprising exposing the array to conditions for removing bound target molecules from the second population from the array.

23. The method of claim 22, wherein the array further comprises second stripping control features arranged in a second pattern different from the first pattern produced by the disposition of the first stripping control features, the second stripping control features for binding to second stripping control target molecules in the second population of target molecules, and the method further comprises detecting the second pattern after exposing bound target molecules from the second population to stripping conditions.

24. The method of claim 20, wherein the first and second pattern comprise symbols representing the order in which target populations of molecules have been contacted with the array.

25. The method of claim 24, wherein the symbols comprise numbers.

26. The method of claim 1, wherein the target molecules comprise biopolymers.

27. The method of claim 1, wherein the target molecules comprise nucleic acid molecules.

28. The method of claim 27, wherein the target molecules comprise genomic DNA.

29. The method of claim 28, wherein the target DNA is non-reduced complexity genomic DNA.

30. The method of claim 28, wherein the target DNA is reduced complexity DNA.

31. The method of claim 28, wherein the target DNA is enriched for DNA that binds to a DNA binding protein.

32. The method of claim 27, wherein the target molecules comprise RNA, cDNA or cRNA.

33. The method of claim 32, wherein the target molecules are enriched for molecules that bind to an RNA binding protein.

34. The method of claim 26, wherein the target molecules comprise polypeptides.

35. A kit comprising an array comprising a plurality of features and a first set of stripping control targets for binding to a first pattern of control probe features on the array.

36. The kit of claim 35, further comprising a second set of stripping control target molecules for binding to a second pattern of stripping control probe features on the array.

37. The kit of claim 36, wherein the first and second pattern are different.

38. The kit of claim 35, wherein the first pattern comprises a symbol.

39. The kit of claim 38, wherein the first pattern comprises a number or character.

40. The kit of claim 37, wherein the first and second pattern comprises different numbers.

41. An array comprising an identifier, wherein the identifier is associated with data relating to stripping conditions to which the array has been or should be exposed to.

42. The array of claim 41, wherein the identifier is associated data relating to the disposition of stripping control probes for validating the efficacy of a stripping procedure on the array.

43. The array of claim 41, wherein the identifier comprises a data element comprising a remotely programmable memory.

44. The array of claim 41, wherein the identifier comprises a bar code tag.

45. A device comprising one or more chambers comprising a means for exposing an array substrate to stripping conditions for removing target molecules bound to probe molecules on the array, wherein the device further comprises or is associated with an identifier reader for reading an identifier on an array.

46. The device of claim 45, wherein the means for exposing comprises an inlet in the chamber which communicates with a reservoir comprising a fluid for stripping the array.

47. The device of claim 45, wherein the means comprises a heating element for raising the temperature of a fluid within the chamber to a temperature effective for stripping the array.

48. The device of claim 45, wherein the chamber further comprises an outlet for removing a fluid from the chamber.

49. The device of claim 45, wherein the chamber comprises an additional inlet for introducing fluids for washing unbound target molecules from an array or introducing target molecules for contacting with the array.

50. The device of claim 45, wherein the chamber further comprises an inlet for introducing a liquid or gaseous fluid for drying the array.

51. The device of claim 45 further comprising an additional chamber comprising an inlet for introducing fluids for washing unbound target molecules from the array or for introducing target molecules for contacting with the array.

52. The device of claim 51, further comprising a means for moving an array substrate from one chamber to another.

53. The device of claim 45, wherein the device comprises a processor for controlling movements of fluids and/or for altering conditions within the one or more chambers.

54. The device of claim 45, wherein a chamber is removable from the device and configured to be placed in a scanner.

55. The device of claim 45, wherein the chamber is configured for receiving an array holder for containing the array substrate.

56. The device of claim 55, wherein the array holder is configured to be placed in a scanner for reading the array.

57. The device of 45, wherein the device comprises a plurality of inlets which communicate independently with one or more chambers of the device.

58. The device of claim 53, wherein the processor communicates with a memory for storing data relating to stripping conditions.

59. The device of claim 58, wherein the device further comprises a user interface for communicating with the processor for displaying data relating to one or more stripping procedures.

60. The device of claim 59, wherein the stripping procedure is associated with an assay type and can be executed by the device in response to a user selecting the assay type.

61. The device of claim 53, wherein the processor receives input from a scanner relating to signal intensity at stripping control features after a stripping procedure and can implement a protocol for re-exposing an array to stripping conditions based on the signal intensity at the control features.

62. The device of claim 53, wherein in response to reading an identifier on the array, the device executes a procedure associated with the identifier in a memory accessed by the processor.

63. The device of claim 53, wherein in response to reading an identifier on the array, the device provides an output relating to procedures to which the array has been exposed within the device.

64. The device of claim 63, wherein the output is displayed on a user interface in communication with the device.

65. The device of claim 63, wherein the user interface is remote from the device.

66. The device of claim 53, wherein the processor accesses a memory storing data relating to array identifiers.

67. The device of claim 66, wherein the data comprises: the nature of probes on the array, a pattern in which one or more stripping control probe features is disposed, the disposition of performance control probes on the array, the ratio of performance control target molecules in a sample, the identity or function of a gene or an encoded product to which the probe corresponds, the nature of a procedure or condition to which the array has been or should be exposed, binding conditions to which the array has been exposed, washing conditions to which the array has been exposed, and/or stripping conditions to which the array has been exposed.

68. The device of claim 67, wherein updated data is provided to the processor prior to and/or after exposing the array to a fluid in one or more chambers.

69. The device of claim 62, wherein when a user selects a procedure to be executed by the device or alters parameters of a procedure to which an array is exposed, information relating to that altered procedure and/or parameters is automatically stored in a memory accessible by the processor and is associated with an identifier on the array.

70. A computer program product comprising instructions for executing operations of a reaction chamber in which an array is being stripped, based on inputted data relating to a pattern of stripping control target molecules bound to stripping control probe features on the array which is exposed to a stripping procedure.

71. The computer program product of claim 70, wherein the product comprises instructions to compute performance of the array based on signal from performance control target molecules hybridized to performance control probes on an array which is exposed to a stripping procedure.

72. The computer program product of claim 70, wherein the product accesses inputs relating to one or more of: stripping conditions, hybridization conditions, and/or washing conditions, and associates the conditions with a value for a signal associated with stripping control probe: stripping control target complexes obtained after stripping and, optionally with a value for a signal associated with performance control probe: performance control target complexes on the array.

73. A computer memory comprising data associating an identifier on an array with a stripping protocol to which the array has been or should be exposed.

74. The memory of claim 73, wherein a processor communicates with the memory to execute instructions relating to the stripping protocol.

75. The method of claim 1, further comprising detecting a binding of performance control target molecules to performance control features on the array to monitor array performance after stripping.
